(19) United States
(12) Patent Application Publication
(10) Pub. No.: US 2025/0236892 A1
(43) Pub. Date: Jul. 24, 2025

(54) Inhibition of NHEJ, MMEJ, and 53BP1 Promotes High Levels of HDR

(71) Applicant: Integrated DNA Technologies, Inc., Coralville, IA (US)

(72) Inventors: Steve Ehren GLENN, North Liberty, IA (US); Vaughn THADA, Coralville, IA (US); Christopher Anthony VAKULSKAS, North Liberty, IA (US)

(73) Assignee: Integrated DNA Technologies, Inc., Coralville, IA (US)

(21) Appl. No.: 19/033,845

(22) Filed: Jan. 22, 2025

Related U.S. Application Data
(60) Provisional application No. 63/623,394, filed on Jan. 22, 2024.

(51) Int. Cl. C12N 15/90; C07D 401/04; C07K 14/47; C12N 9/22; C12N 15/11

(52) U.S. Cl. CPC C12N 15/907; C07D 401/04; C07K 14/4702; C12N 9/22; C12N 15/111; C12N 2310/20

(57) ABSTRACT

The present disclosure pertains to compositions, methods, and kits for improving the rate of double-strand break (DSB) repair by homology directed repair (HDR) through inhibition of 53BP1 mediated DNA end protection in combination with inhibition of microhomology-mediated end joining (MMEJ), or alternatively in combination with inhibition of non-homologous end joining (NHEJ), or in combination with inhibition of both MMEJ and NHEJ.

This application claims priority to U.S. Ser. No. 63/623,394 filed Jan. 22, 2024, the entirety of which is incorporated herein by reference.

### REFERENCE TO AN ELECTRONIC SEQUENCE LISTING

The contents of the electronic sequence listing (6391-0020US01_sequence.xml; Size: 87 kbytes; and Date of Creation: Jan. 16, 2025) is herein incorporated by reference in its entirety.

### FIELD OF THE INVENTION

The compounds and methods as disclosed herein pertain to the ability to improve the rate of double-strand break (DSB) repair by homology directed repair (HDR) through inhibition of 53BP1 mediated DNA end protection in combination with inhibition of microhomology-mediated end joining (MMEJ), or MMEJ and non-homologous end joining (NHEJ).

### BACKGROUND OF THE INVENTION

Double-strand breaks (DSBs) are predominantly repaired through two mechanisms, non-homologous end joining (NHEJ), in which broken ends are rejoined, often imprecisely, or homology directed repair (HDR), where DNA with homology to the break site is used as a template for repair. Typically, the sister chromatid or homologous chromosome is used as the template to facilitate primarily error-free repair. As HDR is facilitated by the presence of a sister chromatid, there are multiple mechanisms in place restricting HDR to the S and G2 phases of the cell cycle¹. NHEJ can occur in G1, S, and G2, however it is most prevalent during the G1 phase of the cell cycle². In addition to HDR and NHEJ, DSBs can also be repaired through an intrinsically mutagenic process called microhomology-mediated end joining (MMEJ). MMEJ involves limited end resection allowing annealing of micro-homologous sequences flanking the DNA ends, typically resulting in larger deletions than with NHEJ repair with the prevalence of MMEJ repair varying depending on the sequence context of the DSB³⁻⁷. Genome editing often involves using HDR to introduce desired edits at a targeted double-strand break. A DNA template containing sequences matching the region around the break flanking a desired edit can be introduced into cells during genome editing as a donor template for repair by HDR. By improving rates of HDR, the compounds and methods as disclosed herein facilitate successful genome editing.

### BRIEF SUMMARY OF THE INVENTION

The disclosure provides a composition for enhancing homology-directed repair (HDR) editing activity in a cell comprising: a. a first recombination enhancing agent that inhibits 53BP1-mediated DNA end protection; and b. at least one additional recombination enhancing agent selected from the group consisting of a second recombination enhancing agent which inhibits microhomology-mediated end joining (MMEJ) and a third recombination enhancing agent which inhibits non-homologous end joining (NHEJ), or combinations thereof. The disclosure provides a composition for enhancing homology-directed repair (HDR) editing activity in a cell wherein the first recombination enhancing agent which inhibits 53BP1-mediated DNA end protection is a 53BP1 binding ubiquitin variant. The disclosure provides a composition for enhancing homology-directed repair (HDR) editing activity in a cell wherein the first recombination enhancing agent which inhibits 53BP1-mediated DNA end protection is a 53BP1 binding ubiquitin variant comprises Ubv-A or any variants thereof. In some embodiments, the 53BP1 binding ubiquitin variant is selected from the group consisting of a Ubv-A polypeptide; an mRNA encoding a Ubv-A polypeptide; and a nucleic acid encoding a Ubv-A polypeptide. The disclosure provides a composition for enhancing homology-directed repair (HDR) editing activity in a cell wherein the first recombination enhancing agent which inhibits 53BP1-mediated DNA end protection is selected from the group consisting of a Ubv-A polypeptide comprising a sequence having 90%, 95%, or 100% identity to SEQ ID NO: 1; an mRNA encoding a Ubv-A polypeptide comprising a sequence having 90%, 95%, or 100% identity to SEQ ID NO: 3; and a nucleic acid encoding a Ubv-A polypeptide comprising a sequence having 90%, 95%, or 100% identity to SEQ ID NO: 2. The disclosure provides a composition for enhancing homology-directed repair (HDR) editing activity in a cell wherein the second recombination enhancing agent which inhibits microhomology-mediated end joining (MMEJ) is selected from the group consisting of an ART558 molecule, RP-6685, Novobiocin, PolQi1, and PolQi2. The disclosure provides a composition for enhancing homology-directed repair (HDR) editing activity in a cell wherein the third recombination enhancing agent which inhibits non-homologous end joining (NHEJ) is selected from the group consisting of NU7441, NU7026, KU-0060648, M3814, CC-115, SCR7, AZD7648 and an Alt-R HDR enhancer V2 molecule. The disclosure provides a composition for enhancing homology-directed repair (HDR) editing activity in a cell further comprising a compound selected from 53BP1 inhibitors, HDAC inhibitors, and combinations thereof. The disclosure provides a composition for enhancing homology-directed repair (HDR) editing activity in a cell wherein the 53BP1 inhibitor is selected from the group consisting of inhibitors of proteins involved in 53BP1 mediated end-protection including but not limited to 53BP1, MDC1, RNF8, RNF168, RIF1, PTIP, Artemis, REV7, SHLD1, SHLD2, SHLD3, CTC1, STN1, TEN1, and combinations thereof. The disclosure provides a composition for enhancing homology-directed repair (HDR) editing activity in a cell wherein the HDAC inhibitor is selected from the group consisting of Romidepsin, Trichostatin A (TSA), and combinations thereof. The disclosure provides a composition for enhancing homology-directed repair (HDR) editing activity in a cell further comprising a CRISPR-associated protein comprising Cas9, Cas12, or any variants thereof. In some embodiments, the CRISPR-associated protein is selected from the group consisting of Cas9 wild-type, a Cas9 variant, SpCas9, Cas12a wild-type, a Cas12 variant, AsCas12a, AsCas12a Ultra, ErCas12a, EURECA-V, and combinations thereof. The disclosure provides a composition for enhancing homology-directed repair (HDR) editing activity in a cell further comprising a nucleic acid encoding a CRISPR-associated protein selected from the group consisting of Cas9 wild-type, a Cas9 variant, SpCas9, Cas12a wild-type, a Cas12 variant, AsCas12a, AsCas12a Ultra, ErCas12a, and combinations thereof. In some embodiments, the CRISPR-associated protein comprises at least one nuclear localization signal (NLS). The disclosure provides a composition for enhancing homology-directed repair (HDR) editing activity in a cell further comprising a guide RNA selected from a single guide RNA (sgRNA) or a combination of a CRISPR RNA (crRNA) and a trans-activating CRISPR RNA (tracrRNA). The disclosure provides a composition for enhancing homology-directed repair (HDR) editing activity in a cell further comprising a donor DNA template. The disclosure provides a composition for enhancing homology-directed repair (HDR) editing activity in a cell wherein the donor DNA template is a single-stranded or double-stranded oligonucleotide. The disclosure provides a composition for enhancing homology-directed repair (HDR) editing activity in a cell wherein the cell is a mammalian cell, a plant cell, a bacterial cell, or a yeast cell. The disclosure provides a composition for enhancing homology-directed repair (HDR) editing activity in a cell wherein the mammalian cell is a human cell. The disclosure provides a composition for enhancing homology-directed repair (HDR) editing activity in a cell wherein the composition for enhancing homology-directed repair (HDR) editing activity in a cell is delivered to the cell using one or more of the following methods: electroporation, microinjection, lipid-based transfection, viral transduction, or nanoparticle delivery. The disclosure provides a kit for enhancing homology-directed repair (HDR) activity in a cell, the kit comprising: a. a first recombination enhancing agent that inhibits 53BP1-mediated DNA end protection; and b. at least one additional recombination enhancing agent selected from the group consisting of a second recombination enhancing agent which inhibits microhomology-mediated end joining (MMEJ) and a third recombination enhancing agent which inhibits non-homologous end joining (NHEJ), or combinations thereof, wherein the kit further comprises instructions for enhancing homology-directed repair (HDR) activity in a cell. The disclosure provides a kit for enhancing homology-directed repair (HDR) activity in a cell wherein the first recombination enhancing agent which inhibits 53BP1-mediated DNA end protection is a 53BP1 binding ubiquitin variant. The disclosure provides a kit for enhancing homology-directed repair (HDR) activity in a cell wherein the first recombination enhancing agent which inhibits 53BP1-mediated DNA end protection is a 53BP1 binding ubiquitin variant comprising Ubv-A or any variants thereof. In some embodiments, the 53BP1 binding ubiquitin variant is selected from the group consisting of a Ubv-A polypeptide; an mRNA encoding a Ubv-A polypeptide; and a nucleic acid encoding a Ubv-A polypeptide. The disclosure provides a kit for enhancing homology-directed repair (HDR) activity in a cell wherein the first recombination enhancing agent which inhibits 53BP1-mediated DNA end protection is selected from the group consisting of a Ubv-A polypeptide comprising a sequence having 90%, 95%, or 100% identity to SEQ ID NO: 1; an mRNA encoding a Ubv-A polypeptide comprising a sequence having 90%, 95%, or 100% identity to SEQ ID NO: 3; and a nucleic acid encoding a Ubv-A polypeptide represented by SEQ ID NO: 2. The disclosure provides a kit for enhancing homology-directed repair (HDR) activity in a cell wherein the second recombination enhancing agent which inhibits microhomology-mediated end joining (MMEJ) is selected from the group consisting of an ART558 molecule, RP-6685, Novobiocin, PolQi1, and PolQi2. The disclosure provides a kit for enhancing homology-directed repair (HDR) activity in a cell wherein the third recombination enhancing agent which inhibits non-homologous end joining (NHEJ) is selected from the group consisting of NU7441, NU7026, KU-0060648, M3814, CC-115, SCR7, AZD7648 and an Alt-R HDR enhancer V2 molecule. The disclosure provides a kit for enhancing homology-directed repair (HDR) activity in a cell further comprising a compound selected from 53BP1 inhibitors, HDAC inhibitors, and combinations thereof. The disclosure provides a kit for enhancing homology-directed repair (HDR) activity in a cell wherein the 53BP1 inhibitor is selected from the group consisting of inhibitors of proteins involved in 53BP1 mediated end-protection including but not limited to 53BP1, MDC1, RNF8, RNF168, RIF1, PTIP, Artemis, REV7, SHLD1, SHLD2, SHLD3, CTC1, STN1, TEN1, and combinations thereof. The disclosure provides a kit for enhancing homology-directed repair (HDR) activity in a cell wherein the HDAC inhibitor is selected from the group consisting of Romidepsin, Trichostatin A (TSA), and combinations thereof. The disclosure provides a kit for enhancing homology-directed repair (HDR) activity in a cell further comprising a CRISPR-associated protein comprising Cas9, Cas12, or any variants thereof. In some embodiments, the CRISPR-associated protein is selected from the group consisting of Cas9 wild-type, a Cas9 variant, SpCas9, Cas12a wild-type, a Cas12 variant, AsCas12a, AsCas12a Ultra, ErCas12a, EURECA-V, and combinations thereof. The disclosure provides a kit for enhancing homology-directed repair (HDR) activity in a cell further comprising a nucleic acid encoding a CRISPR-associated protein comprising Cas9, Cas12, or any variants thereof or the CRISPR-associated protein selected from the group consisting of Cas9 wild-type, a Cas9 variant, SpCas9, Cas12a wild-type, a Cas12 variant, AsCas12a, AsCas12a Ultra, ErCas12a, EURECA-V, and combinations thereof. In some embodiments, the CRISPR-associated protein comprises at least one nuclear localization signal (NLS). The disclosure provides a kit for enhancing homology-directed repair (HDR) activity in a cell further comprising a guide RNA selected from a single guide RNA (sgRNA) or a combination of a CRISPR RNA (crRNA) and a trans-activating CRISPR RNA (tracrRNA). The disclosure provides a kit for enhancing homology-directed repair (HDR) activity in a cell further comprising a donor DNA template. The disclosure provides a kit for enhancing homology-directed repair (HDR) activity in a cell wherein the donor DNA template is a single-stranded or double-stranded oligonucleotide. The disclosure provides a kit for enhancing homology-directed repair (HDR) activity in a cell wherein the cell is a mammalian cell, a plant cell, a bacterial cell, or a yeast cell. The disclosure provides a kit for enhancing homology-directed repair (HDR) activity in a cell wherein the mammalian cell is a human cell. The disclosure provides a kit for enhancing homology-directed repair (HDR) activity in a cell wherein the composition for enhancing homology-directed repair (HDR) editing activity in a cell is delivered to the cell using one or more of the following methods: electroporation, microinjection, lipid-based transfection, viral transduction, or nanoparticle delivery.

The disclosure provides a method for enhancing homology-directed repair (HDR) editing activity in a cell, the method comprising: a. introducing into the cell a first recombination enhancing agent that inhibits 53BP1-mediated DNA end protection; and b. introducing into the cell at least one additional recombination enhancing agent selected from the group consisting of a second recombination enhancing agent which inhibits microhomology-mediated end joining (MMEJ) and a third recombination enhancing agent which inhibits non-homologous end joining (NHEJ), or combinations thereof, wherein the presence of the at least one recombination enhancing agent increases HDR editing activity at the targeted genomic locus relative to HDR editing activity at the targeted genomic locus in the absence of the at least one recombination enhancing agent. The disclosure provides a method for enhancing homology-directed repair (HDR) editing activity in a cell wherein the first recombination enhancing agent which inhibits 53BP1-mediated DNA end protection is a 53BP1 binding ubiquitin variant. The disclosure provides a method for enhancing homology-directed repair (HDR) editing activity in a cell wherein the first recombination enhancing agent which inhibits 53BP1-mediated DNA end protection is a 53BP1 binding ubiquitin variant comprising Ubv-A or any variants thereof. In some embodiments, the 53BP1 binding ubiquitin variant is selected from the group consisting of a Ubv-A polypeptide; an mRNA encoding a Ubv-A polypeptide; and a nucleic acid encoding a Ubv-A polypeptide. The disclosure provides a method for enhancing homology-directed repair (HDR) editing activity in a cell wherein the first recombination enhancing agent which inhibits 53BP1-mediated DNA end protection is selected from the group consisting of a Ubv-A polypeptide comprising a sequence having 90%, 95%, or 100% identity to SEQ ID NO: 1; an mRNA encoding a Ubv-A polypeptide comprising a sequence having 90%, 95%, or 100% identity to SEQ ID NO: 3; and a nucleic acid encoding a Ubv-A polypeptide comprising a sequence having 90%, 95%, or 100% identity to SEQ ID NO: 2. The disclosure provides a method for enhancing homology-directed repair (HDR) editing activity in a cell wherein the second recombination enhancing agent which inhibits microhomology-mediated end joining (MMEJ) is selected from the group consisting of an ART558 molecule, RP-6685, Novobiocin, PolQi1, and PolQi2. The disclosure provides a method for enhancing homology-directed repair (HDR) editing activity in a cell wherein the third recombination enhancing agent which inhibits non-homologous end joining (NHEJ) is selected from the group consisting of NU7441, NU7026, KU-0060648, M3814, CC-115, SCR7, AZD7648 and an Alt-R HDR enhancer V2 molecule. The disclosure provides a method for enhancing homology-directed repair (HDR) editing activity in a cell further comprising introducing into the cell a compound selected from 53BP1 inhibitors, HDAC inhibitors, and combinations thereof. The disclosure provides a method for enhancing homology-directed repair (HDR) editing activity in a cell wherein the 53BP1 inhibitor is selected from the group consisting of inhibitors of proteins involved in 53BP1 mediated end-protection including but not limited to 53BP1, MDC1, RNF8, RNF168, RIF1, PTIP, Artemis, REV7, SHLD1, SHLD2, SHLD3, CTC1, STN1, TEN1, and combinations thereof. The disclosure provides a method for enhancing homology-directed repair (HDR) editing activity in a cell wherein the HDAC inhibitor is selected from the group consisting of Romidepsin, Trichostatin A (TSA), and combinations thereof. The disclosure provides a method for enhancing homology-directed repair (HDR) editing activity in a cell further comprising introducing into the cell a CRISPR-associated protein comprising Cas9, Cas12, or any variants thereof. In some embodiments, the CRISPR-associated protein is selected from the group consisting of Cas9 wild-type, a Cas9 variant, SpCas9, Cas12a wild-type, a Cas12 variant, AsCas12a, AsCas12a Ultra, ErCas12a, EURECA-V, and combinations thereof. The disclosure provides a method for enhancing homology-directed repair (HDR) editing activity in a cell further comprising introducing into the cell a nucleic acid encoding a CRISPR-associated protein comprising Cas9, Cas12, or any variants thereof. In some embodiments, the CRISPR-associated protein is selected from the group consisting of Cas9 wild-type, a Cas9 variant, SpCas9, Cas12a wild-type, a Cas12 variant, AsCas12a, AsCas12a Ultra, ErCas12a, EURECA-V and combinations thereof. In some embodiments, the CRISPR-associated protein comprises at least one nuclear localization signal (NLS). The disclosure provides a method for enhancing homology-directed repair (HDR) editing activity in a cell further comprising introducing into the cell a guide RNA selected from a single guide RNA (sgRNA) or a combination of a CRISPR RNA (crRNA) and a trans-activating CRISPR RNA (tracrRNA). The disclosure provides a method for enhancing homology-directed repair (HDR) editing activity in a cell further comprising introducing into the cell a donor DNA template. The disclosure provides a method for enhancing homology-directed repair (HDR) editing activity in a cell wherein the donor DNA template is a single-stranded or double-stranded oligonucleotide. The disclosure provides a method for enhancing homology-directed repair (HDR) editing activity in a cell wherein the cell is a mammalian cell, a plant cell, a bacterial cell, or a yeast cell. The disclosure provides a method for enhancing homology-directed repair (HDR) editing activity in a cell wherein the mammalian cell is a human cell. The disclosure provides a method for enhancing homology-directed repair (HDR) editing activity in a cell wherein the method further comprises monitoring HDR activity by detecting the incorporation of the donor DNA at the targeted genomic locus. The disclosure provides a method for enhancing homology-directed repair (HDR) editing activity in a cell wherein the introduction of the effective amount of at least one recombination enhancing agent is performed concurrently with or sequentially to the introduction of the Cas protein, guide RNA, and donor DNA template. The disclosure provides a method for enhancing homology-directed repair (HDR) editing activity in a cell further comprising introducing into the cell a CRISPR-associated protein, wherein the first recombination enhancing agent that inhibits 53BP1-mediated DNA end protection and at least one additional recombination enhancing agent are introduced concurrently with the CRISPR-associated protein into the cell. The disclosure provides a method for enhancing homology-directed repair (HDR) editing activity in a cell wherein the first recombination enhancing agent that inhibits 53BP1-mediated DNA end protection and at least one additional recombination enhancing agent and the CRISPR editing system is delivered to the cell using one or more of the following methods: electroporation, microinjection, lipid-based transfection, viral transduction, or nanoparticle delivery.

The disclosure provides a method for improving the rate of double-strand break (DSB) repair by homology directed repair (HDR) at a DNA target site in a cell by a targeting DNA endonuclease, the method comprising: introducing into the cell genome editing agents and recombination enhancing agents; wherein the genome editing agents comprise the targeting DNA endonuclease and a donor DNA, wherein the recombination enhancing agents comprise a first component, a second component, and optionally a third component, wherein the first component inhibits 53BP1-mediated DNA end protection, the second component inhibits microhomology-mediated end joining (MMEJ), and the third component inhibits non-homologous end joining (NHEJ), wherein the rate of double-strand break (DSB) repair by homology directed repair (HDR) at the DNA target site by the targeting DNA endonuclease is improved.

The disclosure provides a method for improving the rate of double-strand break (DSB) repair by homology directed repair (HDR) at a DNA target site in a cell by a targeting DNA endonuclease wherein the first component comprises a Ubv-A molecule, the second component comprises a MMEJ inhibitor ART558 molecule, and the third component comprises a NHEJ inhibitor Alt-R HDR enhancer V2 molecule. The disclosure provides a method for improving the rate of double-strand break (DSB) repair by homology directed repair (HDR) at a DNA target site in a cell by a targeting DNA endonuclease wherein the Ubv-A molecule is selected from a Ubv-A polypeptide or a nucleic acid encoding a Ubv-A polypeptide. The disclosure provides a method for improving the rate of double-strand break (DSB) repair by homology directed repair (HDR) at a DNA target site in a cell by a targeting DNA endonuclease wherein the nucleic acid encoding a Ubv-A polypeptide is selected from a mRNA encoding a Ubv-A polypeptide, or a DNA encoding a Ubv-A polypeptide. The disclosure provides a method for improving the rate of double-strand break (DSB) repair by homology directed repair (HDR) at a DNA target site in a cell by a targeting DNA endonuclease wherein the targeting DNA endonuclease comprises a Clustered Regularly Interspaced Short Palindromic Repeat (CRISPR) endonuclease. The disclosure provides a method for improving the rate of double-strand break (DSB) repair by homology directed repair (HDR) at a DNA target site in a cell by a targeting DNA endonuclease wherein the Clustered Regularly Interspaced Short Palindromic Repeat (CRISPR)-associated protein comprises Cas9, Cas12, or any variants thereof. In some embodiments, the CRISPR-associated protein is selected from the group consisting of Cas9 wild-type, a Cas9 variant, SpCas9, Cas12a wild-type, a Cas12 variant, AsCas12a, AsCas12a Ultra, ErCas12a, EURECA-V, and combinations thereof. In some embodiments, the CRISPR-associated protein comprises at least one nuclear localization signal (NLS). The disclosure provides a method for improving the rate of double-strand break (DSB) repair by homology directed repair (HDR) at a DNA target site in a cell by a targeting DNA endonuclease wherein the Clustered Regularly Interspaced Short Palindromic Repeat (CRISPR) endonuclease is supplied as a Ribonucleoprotein complex comprising a CRISPR polypeptide and a suitable guide RNA, or as a mixture of nucleic acids encoding CRISPR polypeptide and a suitable guide RNA.

The disclosure provides a method for improving the rate of homology directed repair (HDR) of a DNA endonuclease induced double-strand break (DSB) at a DNA target site in a cell, the method comprising: introducing into the cell genome editing agents and recombination enhancing agents; wherein the genome editing agents comprise the targeting DNA endonuclease and a donor DNA, wherein the recombination enhancing agents comprise a first component that inhibits 53BP1-mediated DNA end protection and a second component that inhibits non-homologous end joining (NHEJ), wherein the rate of double-strand break (DSB) repair by homology directed repair (HDR) at a DNA target site by a DNA endonuclease is improved. The disclosure provides a method for improving the rate of double-strand break (DSB) repair by homology directed repair (HDR) at a DNA target site in a cell by a DNA endonuclease wherein the first component comprises a Ubv-A molecule and the second component comprises a NHEJ inhibitor Alt-R HDR enhancer V2 molecule. The disclosure provides a method for improving the rate of double-strand break (DSB) repair by homology directed repair (HDR) at a DNA target site in a cell by a DNA endonuclease wherein the Ubv-A molecule is selected from a Ubv-A polypeptide or a nucleic acid encoding a Ubv-A polypeptide. The disclosure provides a method for improving the rate of double-strand break (DSB) repair by homology directed repair (HDR) at a DNA target site in a cell by a DNA endonuclease wherein the nucleic acid encoding a Ubv-A polypeptide is selected from a mRNA encoding a Ubv-A polypeptide, or a DNA encoding a Ubv-A polypeptide. The disclosure provides a method for improving the rate of double-strand break (DSB) repair by homology directed repair (HDR) at a DNA target site in a cell by a DNA endonuclease wherein the targeting DNA endonuclease comprises a Clustered Regularly Interspaced Short Palindromic Repeat (CRISPR) endonuclease. The disclosure provides a method for improving the rate of double-strand break (DSB) repair by homology directed repair (HDR) at a DNA target site in a cell by a DNA endonuclease wherein the Clustered Regularly Interspaced Short Palindromic Repeat (CRISPR)-associated protein comprises Cas9, Cas12, or any variants thereof. In some embodiments, the CRISPR-associated protein is selected from the group consisting of Cas9 wild-type, a Cas9 variant, SpCas9, Cas12a wild-type, a Cas12 variant, AsCas12a, AsCas12a Ultra, ErCas12a, EURECA-V, and combinations thereof. In some embodiments, the CRISPR-associated protein comprises at least one nuclear localization signal (NLS). The disclosure provides a method for improving the rate of double-strand break (DSB) repair by homology directed repair (HDR) at a DNA target site in a cell by a DNA endonuclease wherein the Clustered Regularly Interspaced Short Palindromic Repeat (CRISPR) endonuclease is supplied as a Ribonucleoprotein complex comprising a CRISPR polypeptide and a suitable guide RNA or as a mixture of nucleic acids encoding CRISPR polypeptide and a suitable guide RNA.

The disclosure provides a kit for use in improving the rate of double-strand break (DSB) repair by homology directed repair (HDR) at a DNA target site by a targeting DNA endonuclease, the kit comprising: a recombination enhancing agent that improves the rate of double-strand break repair by homology directed repair at the DNA target site by the targeting DNA endonuclease, wherein the recombination enhancing agent comprises a member selected from the group consisting of an inhibitor of 53BP1-mediated DNA end protection; an inhibitor of microhomology-mediated end joining (MMEJ); an inhibitor of non-homologous end joining (NHEJ); or a combination thereof. The disclosure provides a kit for use in improving the rate of double-strand break (DSB) repair by homology directed repair (HDR) at a DNA target site by a targeting DNA endonuclease wherein the recombination enhancing agent comprises the inhibitor of 53BP1-mediated DNA end protection; and a member selected from the group consisting of the inhibitor of microhomology-mediated end joining (MMEJ); and the inhibitor of non-homologous end joining (NHEJ). The disclosure provides a kit for use in improving the rate of double-strand break (DSB) repair by homology directed repair (HDR) at a DNA target site by a targeting DNA endonuclease wherein the recombination enhancing agent comprises the inhibitor of 53BP1-mediated DNA end protection; and the inhibitor of microhomology-mediated end joining (MMEJ). The disclosure provides a kit for use in improving the rate of double-strand break (DSB) repair by homology directed repair (HDR) at a DNA target site by a targeting DNA endonuclease wherein the recombination enhancing agent comprises the inhibitor of 53BP1-mediated DNA end protection; and the inhibitor of non-homologous end joining (NHEJ). The disclosure provides a kit for use in improving the rate of double-strand break (DSB) repair by homology directed repair (HDR) at a DNA target site by a targeting DNA endonuclease wherein the inhibitor of 53BP1-mediated DNA end protection comprises a Ubv-A molecule, the inhibitor of microhomology-mediated end joining (MMEJ) comprises the MMEJ inhibitor ART558 molecule, and the inhibitor of non-homologous end joining (NHEJ). comprises the NHEJ inhibitor Alt-R HDR enhancer V2 molecule. The disclosure provides a kit for use in improving the rate of double-strand break (DSB) repair by homology directed repair (HDR) at a DNA target site by a targeting DNA endonuclease wherein the Ubv-A molecule is selected from a Ubv-A polypeptide or a nucleic acid encoding a Ubv-A polypeptide. The disclosure provides a kit for use in improving the rate of double-strand break (DSB) repair by homology directed repair (HDR) at a DNA target site by a targeting DNA endonuclease wherein the nucleic acid encoding a Ubv-A polypeptide is selected from a mRNA encoding a Ubv-A polypeptide, or a DNA encoding a Ubv-A polypeptide. The disclosure provides a kit for use in improving the rate of double-strand break (DSB) repair by homology directed repair (HDR) at a DNA target site by a targeting DNA endonuclease further comprising the targeting DNA endonuclease. The disclosure provides a kit for use in improving the rate of double-strand break (DSB) repair by homology directed repair (HDR) at a DNA target site by a targeting DNA endonuclease wherein the targeting DNA endonuclease comprises a Clustered Regularly Interspaced Short Palindromic Repeat (CRISPR) endonuclease. The disclosure provides a kit for use in improving the rate of double-strand break (DSB) repair by homology directed repair (HDR) at a DNA target site by a targeting DNA endonuclease wherein the Clustered Regularly Interspaced Short Palindromic Repeat (CRISPR)-associated protein comprising Cas9, Cas12, or any variants thereof. In some embodiments, the CRISPR-associated protein is selected from the group consisting of Cas9 wild-type, a Cas9 variant, SpCas9, Cas12a wild-type, a Cas12 variant, AsCas12a, AsCas12a Ultra, ErCas12a, EURECA-V, and combinations thereof. In some embodiments, the CRISPR-associated protein comprises at least one nuclear localization signal (NLS). The disclosure provides a kit for use in improving the rate of double-strand break (DSB) repair by homology directed repair (HDR) at a DNA target site by a targeting DNA endonuclease wherein the Clustered Regularly Interspaced Short Palindromic Repeat (CRISPR) endonuclease is supplied as a Ribonucleoprotein complex comprising a CRISPR polypeptide and a suitable guide RNA or as a mixture of nucleic acids encoding CRISPR polypeptide and a suitable guide RNA. The disclosure provides a kit for use in improving the rate of double-strand break (DSB) repair by homology directed repair (HDR) at a DNA target site by a targeting DNA endonuclease further comprising the instructions of the method as disclosed herein.

### BRIEF DESCRIPTION OF THE DRAWINGS

FIG. 1 depicts inhibition of NHEJ, MMEJ, and 53BP1 leads to high levels of HDR at SpCas9 induced DSBs. HEK293 cells were treated with Cas9 RNP (2 μM) targeting 5 different sites (FIG. 1A: AADACL3; FIG. 1B: ANKRD50; FIG. 1C: ABCD4: FIG. 1D: AR; FIG. 1E: AKAP6) in the genome alongside ssDNA donor template with or without Ubv-A protein (25 μM). Cells were then plated in media containing no DMSO, DMSO control, ART, V2, or ART+V2 for 24 hours before exchanging for fresh media (see Methods for concentrations). The graphs show the percent of each repair event under each condition measured by NGS with error bars indicating SD from three replicates. Likely MMEJ deletions are determined based on the presence of microhomology of two nucleotides or greater at the ends of the deletion.

FIG. 2 depicts inhibition of NHEJ, MMEJ, and 53BP1 leads to high levels of HDR at AsCas12a induced DSBs. HEK293 cells were treated with AsCas12a Ultra RNP (2 μM) targeting 5 different sites (FIG. 2A: SERPINC1; FIG. 2B: HPRT1; FIG. 2C: RAG2: FIG. 2D: AR; FIG. 2E: MET) in the genome alongside ssDNA donor template with or without Ubv-A protein (25 μM). Cells were then plated in media containing no DMSO, DMSO control, ART, V2, or ART+V2 for 24 hours before exchanging for fresh media (see methods section for concentrations). The graphs show the percent of each repair event under each condition measured by NGS with error bars indicating SD from three replicates. Likely MMEJ deletions are determined based on the presence of microhomology of two nucleotides or greater at the ends of the deletion.

FIG. 3A depicts percent CAR knock-in (KI) at the TRAC locus in HEK293 cells treated with 1 μg dsDNA Alt-R HDR donor block, 2 μM Cas9 RNP, and either 25 μM tagless Ubv-A protein or 25 nM Ubv-A mRNA or no Ubv-A and incubated in media containing DMSO (no enhancer), MMEJ inhibitor ART558 (ART, 10 μM), NHEJ inhibitor Alt-R HDR enhancer V2 (V2, 1 μM), or both ART558 and V2. Percent KI was calculated using PCR followed by analysis using an Agilent Fragment Analyzer and data processing using PROSize 3.0 software to calculate the relative molar concentrations between wild-type and edited products. Error bars indicate SD, n=3.

FIG. 3B depicts percent CAR knock-in (KI) at the TRAC locus in HEK293 cells treated with 1 μg dsDNA Alt-R HDR donor block, 1 μg Cas9 mRNA plus 4.8 μM sgRNA, and either 25 μM tagless Ubv-A protein or 25 nM Ubv-A mRNA or no Ubv-A and incubated in media containing DMSO (no enhancer), MMEJ inhibitor ART558 (ART, 10 μM), NHEJ inhibitor Alt-R HDR enhancer V2 (V2, 1 μM), or both ART558 and V2. Percent KI was calculated using PCR followed by analysis using an Agilent Fragment Analyzer and data processing using PROSize 3.0 software to calculate the relative molar concentrations between wild-type and edited products. Error bars indicate SD, n=3.

FIG. 4A depicts that inhibition of NHEJ, MMEJ, and 53BP1 leads to high levels of HDR at EURECA-V induced DSBs. HEK293 cells were treated with EURECA-V RNP (3 μM, Aldevron) targeting the SERPINC1 site in the genome alongside ssDNA donor template (2 μM) with or without Ubv-A protein (25 μM). Cells were then plated in media containing no DMSO, DMSO control, ART558 (ART, 10 M, MedChemExpress HY-141520), Alt-R HDR enhancer V2 (1 μM, IDT), or ART+V2 for 24 hours before exchanging for fresh media. The graphs show the percent of each repair event under each condition measured by NGS with error bars indicating SD from three replicates. Likely MMEJ deletions are determined based on the presence of microhomology of two nucleotides or greater at the ends of the deletion.

FIG. 4B depicts that inhibition of NHEJ, MMEJ, and 53BP1 leads to high levels of HDR at EURECA-V induced DSBs. HEK293 cells were treated with EURECA-V RNP (3 μM, Aldevron) targeting the HPRT1 site in the genome alongside ssDNA donor template (2 μM) with or without Ubv-A protein (25 μM). Cells were then plated in media containing no DMSO, DMSO control, ART558 (ART, 10 μM, MedChemExpress HY-141520), Alt-R HDR enhancer V2 (1 μM, IDT), or ART+V2 for 24 hours before exchanging for fresh media. The graphs show the percent of each repair event under each condition measured by NGS with error bars indicating SD from three replicates. Likely MMEJ deletions are determined based on the presence of microhomology of two nucleotides or greater at the ends of the deletion.

FIG. 4C depicts that inhibition of NHEJ, MMEJ, and 53BP1 leads to high levels of HDR at EURECA-V induced DSBs. HEK293 cells were treated with EURECA-V RNP (3 μM, Aldevron) targeting the AR site in the genome alongside ssDNA donor template (2 μM) with or without Ubv-A protein (25 μM). Cells were then plated in media containing no DMSO, DMSO control, ART558 (ART, 10 μM, MedChemExpress HY-141520), Alt-R HDR enhancer V2 (1 μM, IDT), or ART+V2 for 24 hours before exchanging for fresh media. The graphs show the percent of each repair event under each condition measured by NGS with error bars indicating SD from three replicates. Likely MMEJ deletions are determined based on the presence of microhomology of two nucleotides or greater at the ends of the deletion.

### DETAILED DESCRIPTION OF THE INVENTION

HDR begins with the recruitment of a complex of proteins consisting of MRE11, NBS1, and RAD50 (MRN) to the site of a DSB. MRE11 is an endonuclease that cuts the 5′-terminated strand of DNA distal from the broken DNA end. MRE11 also has 3′ to 5′ exonuclease activity that subsequently digests the DNA towards the direction of the break, resulting in a short 3′ overhang⁸. This resection depends upon interaction of the MRN complex with C terminal binding protein interacting protein (CtIP). CtIP is phosphorylated in a cell cycle dependent manner by CDK to facilitate end resection during HR permissive S and G2 phases of the cell cycle⁸. This limited resection is then extended to long range resection by two redundant nucleases, EXO1 and BLM-DNA2⁹. The ssDNA is stabilized by RPA, a heterotrimeric complex of three tightly associated subunits. RPA is subsequently exchanged for RAD51 in a process mediated by BRCA2⁸. RAD51 facilitates strand invasion and HDR repair using homologous DNA sequences.

Repair of a DSB by NHEJ starts with binding of Ku70 and Ku80 to the broken DNA ends followed by recruitment of DNA dependent protein kinase catalytic subunit (DNA-PKcs). The combination of Ku70/80 and DNA-PKcs form the DNA-PK complex. DNA-PK is thought to bridge the broken DNA ends, protecting them from degradation while bringing them together^{10,11}. DNA-PKcs phosphorylates itself as well as other DNA repair factors to facilitate repair. Broken DNA ends are often not compatible with ligation and require further processing by enzymes such as Artemis, PNKP, and gap-filling DNA polymerases before being ligated by the XRCC4-XLF complex in combination with DNA-ligase IV^{11,12}.

Inhibition of NHEJ is a common approach to improve HDR rates as HDR and NHEJ are competing pathways for repair of a DSB. The most common method of inhibiting NHEJ is through the use of small molecules to inhibit DNA-PKcs kinase activity. Inhibitors of DNA-PKcs that have been shown to improve rates of HDR include NU7441, NU7026, KU-0060648, M3814 (Peposertib), CC-115, SCR7 and AZD7648¹³⁻¹⁶. Similarly, inhibition of the upstream Ku-DNA interaction using Ku-DBi 245 or inhibition of the downstream ligase complex using SCR7 also improves HDR in some contexts^{14, 17-19}. Knockdown of various NHEJ components such Ku70, Ku80, DNA-PKcs, and LIG4 by CRISPRi and DNA-PKcs and DNA ligase IV using siRNA KD also improves HDR^{13,20}.

MMEJ, like HDR, is initiated by limited end resection requiring the MRN complex and CtIP. However, unlike HDR, MMEJ does not require EXO1, or BLM-DNA2, and is inhibited by long range end resection^{6, 21, 22}. After limited end resection, DNA polymerase theta, the MRN complex, and poly (ADP-ribose) polymerase 1 (PARP1) facilitate end-bridging and annealing of short (2-20 bp) homologous sequences in the 3′ overhangs. Nonhomologous 3′ flaps are processed if present and the gaps are filled in. This gap filling is performed by DNA polymerase theta followed by final ligation, which is mediated by DNA ligase 3 or DNA ligase 1 in complex with XRCC1^{5, 6, 22-24}.

The level of HDR, NHEJ, and MMEJ repair of double-strand breaks varies between different target sites in the genome. Local sequence context can influence the ratio of NHEJ to MMEJ repair of a double-strand break allowing some prediction of repair outcomes⁷. Because both NHEJ and MMEJ repair of a break is undesired for HDR mediated genome editing, multiple groups have explored the use of inhibitors of MMEJ alone or in combination with inhibitors of NHEJ^{16,25-27}. Novel MMEJ inhibitors have recently been identified including RP-6685, ART558, Novobiocin, PolQi1, and PolQi2, some of which have been shown to improve HDR or the purity of HDR events alone or in combination with DNA-PKcs inhibitors^{4, 16, 28-32}. RNAi knockdown of DNA pol theta has also been used in combination with a small molecule DNA-PKcs inhibitor, M3814, to improve HDR rates^{25,26}.

A key determinant in repair pathway choice is p53-binding protein 1 (53BP1). 53BP1 was first described as a binding partner of the tumor suppressor gene p53 and was later shown to facilitate NHEJ³³. 53BP1 promotes NHEJ by limiting end resection during G1 when HDR is inactive³⁴. End resection is critical for HDR and resected ends are resistant to NHEJ; therefore, 53BP1 functions as both an HDR suppressor and a promoter of NHEJ by keeping DNA ends compatible with NHEJ³⁵. Inhibition of end resection by 53BP1 requires recruitment of 53BP1 to sites of DSBs. This recruitment is dependent upon both H4K20 methylation and H2AK15 ubiquitination. Ubiquitin is a small, highly conserved protein found in eukaryotic cells that plays a critical role in regulating various cellular processes. It consists of 76 amino acids and serves as a molecular tag for proteins. Ubiquitin is covalently attached to target proteins through an enzymatic process involving three types of enzymes: E1 (activating enzyme), E2 (conjugating enzyme), and E3 (ligase). The attachment of ubiquitin, a process called ubiquitination, marks proteins for specific outcomes, for example, proteasomal degradation, signal transduction, DNA repair, and endocytosis. 53BP1 has a tandem Tudor domain (TTD) that has been shown to specifically bind mono and dimethylated H4K20^{36, 37}. 53BP1 recruitment also requires a short region C-terminal of the TDD containing a ubiquitination-dependent recruitment motif (UDR) that binds to H2AK15ub³⁸. Upon DNA damage, the MRN complex is recruited to broken DNA ends. MRN facilitates recruitment of the protein kinase ATM that phosphorylates a number of different targets including H2AX. Phosphorylation of H2AX results in recruitment of MDC1 which is itself phosphorylated by ATM and then bound by the FHA domain of RNF8, an E3 ubiquitin ligase. RNF8 ubiquitinated H2A is bound by the MIUs (motif interacting with ubiquitin) of RNF168, a second RING type E3 ubiquitin ligase^{39, 40}. Both RNF168 and RNF8 cause degradation of JMJD2A/B and L3MBTL1 that normally bind H4K20me2 and inhibit 53BP1 recruitment^{41, 42}. Further, RNF168 ubiquitinates H2AK15 which is then bound by 53BP1 to facilitate its recruitment⁴³. 53BP1 acts as a tether for the recruitment of PTIP and RIF1 adapter proteins that recruit additional factors to mediate end protection^{44, 45}. RIF1 interacts with the SHLD3 subunit of shieldin, a complex of four proteins (REV7, SHLD1, SHLD2, and SHLD3) thought to be an effector of end protection through ssDNA binding to block further exonuclease mediated resection⁴⁶. Further, interaction of SHLD1 and SHLD3 with CTC1 and STN1, respectively, of the CTC1-STN1-TEN1 (CST) complex also contributes to end protection⁴⁷. This complex is thought to facilitate DNA polymerase a mediated fill-in of the resected ends to limit end resection at double-strand breaks. PTIP interaction with the nuclease Artemis was also shown to suppress HDR in a nuclease activity dependent manner⁴⁸. Loss of 53BP1 recruitment or its downstream effectors results in increased end resection and a bias toward repair of DSBs via HDR^{46, 49-51}.

Multiple approaches have been used to inhibit 53BP1. Multiple small molecule inhibitors of 53BP1 have been identified, including UNC2170 and DP308, that bind to the methyl lysine binding pocket of 53BP1 to inhibit recruitment^{52, 53}. In 2018, Canney et al. identified a variant of human ubiquitin that functions as an inhibitor of 53BP1 (i53) that binds the 53BP1 tudor domain and inhibits 53BP1 recruitment to sites of doubles strand breaks⁴⁹. In an alternative approach, Paulsen et al. used a tudor domain containing fragment of 53BP1 (dn53BP1) lacking end protection activity to block recruitment of the functional full-length 53BP1 to enhance rates of HDR^{54, 55} Overexpression of JMJD2A, which binds H4K20me2 and is degraded by RNF8/RNF168 upon DNA damage, inhibits 53BP1 foci formation⁴¹. Similarly, an engineered variant of RAD18 that binds to H2AK15ub also inhibits 53BP1 recruitment and increases rates of HDR⁵⁶. While these are some of the methods for 53BP1 inhibition that have been used, inhibition or knockdown of factors upstream or downstream of 53BP1 such as MDC1, RNF8, RNF168, RIF1, PTIP, Artemis, components of the Shieldin complex (REV7, SHLD1, SHLD2, SHLD3), or components of the CST complex (CTC1-STN1-TEN1) may similarly promote end resection and increase rates of HDR^{44-48, 57}.

Interestingly, other small molecules commonly found to increase the rate of HDR including HDAC inhibitors such as Romidepsin and Trichostatin A (TSA), as well as Povenedistat, an inhibitor of NEDD8-activating enzyme, may at least partially function to enhance HDR through inhibition of 53BP1 recruitment^{14, 58-61}. H4K16 acetylation disrupts the binding of 53BP1 to H4K20me2⁶². HDAC1 and HDAC2 deacetylate H4K16 following a double-strand break and inhibition of HDAC1 and HDAC2 using TSA reduces recruitment of 53BP1 to chromatin suggesting that HDAC inhibitors may at least partially enhance HDR through limiting 53BP1 recruitment^{62, 63}. Povenedistat/MLN4924 is a neddylation inhibitor that was found to bias repair toward HDR while increasing end resection and decreasing the recruitment of 53BP1 effector RIF1 to DNA breaks⁵⁸. Recruitment of 53BP1 to breaks is dependent upon the E3 ubiquitin ligase RNF168, which is itself dependent on DNA damage induced histone polyneddylation. Loss of DNA-damage induced polyneddylation leads to reduced DNA damage-induced RNF168 and 53BP1 foci⁶⁴. These findings support a model whereby MLN4924 biases the cell towards HDR through inhibition of neddylation resulting in loss of RNF168 recruitment and therefore loss of 53BP1 recruitment resulting in increased end resection.

Due to the affinity of 53BP1 for ubiquitinated H2A, a screen of ubiquitin variants for interaction with 53BP1 was conducted recently by Canny et al. in which they discovered and modified a ubiquitin variant with selective binding to 53BP1 that they named i53 (inhibitor of 53BP1)⁴⁹. Previous filings from IDT described the results of a screen to identify ubiquitin variants (Ubvs) with increased affinity for 53BP1 and improved efficacy for enhancing HDR rates compared to i53. From that screen, and additional targeted engineering, we generated a set of ubiquitin variants that have improved affinity for 53BP1. We demonstrated that one of our engineered variants, Ubv-A, was able to improve rates of HDR in HEK293 cells using ssDNA donor, dsDNA donor, and AAV donor and was able to boost HDR in K562 cells, Jurkat cells, iPSCs, and primary T cells. We also demonstrated that Ubv-A was able to further improve HDR rates when used in conjunction with a small molecule that inhibits NHEJ, Alt-R HDR enhancer V2 (V2).

This disclosure provides the ability for a 53BP1 binding ubiquitin variant, such as Ubv-A, to enhance HDR when used with a variety of nucleases, such as, for example, a 53BP1 binding ubiquitin variant such as Ubv-A when used in combination with, for example, SpCas9, AsCas12a, and/or EURECA-V (a ErCas12a variant). Different mechanisms of boosting HDR can be more or less effective depending on the nature of the DNA break such as whether a break has blunt ends or overhangs⁹. These data demonstrate that a 53BP1 binding ubiquitin variant, such as Ubv-A is effective at boosting HDR to a similar degree whether a cut is blunt, as with SpCas9, or staggered, as with AsCas12a or ErCas12a.

While inhibition of DNA-PKcs is commonly used to improve HDR rates, recent studies have revealed that it can cause large-scale genomic alterations leading to hesitancy in using it for therapuetic applications⁵³. We have similarly found that inhibition of NHEJ results in a large increase in the frequency of translocations and off-target editing in HEK293 cells. In contrast, no increase in translocations or off-target editing was observed with use of a 53BP1 binding ubiquitin variant, such as Ubv-A to increase HDR rates. We also do not observe any decrease in cell viability commonly observed with DNA-PKcs inhibitors. We envision that our ubiquitin variants targeting 53BP1 can be a valuable tool for ex vivo genome editing, where both high efficiency and cell viability are needed and the effects of blocking NHEJ are a potential concern.

CRISPR-associated (Cas) proteins useful in certain embodiments as disclosed herein may include: Cas9: The most well-known CRISPR protein, primarily from Streptococcus pyogenes (SpCas9), which cuts double-stranded DNA with high precision using a single-guide RNA (sgRNA); SaCas9 (Staphylococcus aureus)—smaller than SpCas9, useful for viral delivery; NmCas9 (Neisseria meningitidis)—recognizes a different PAM and offers alternative targeting sites; St1Cas9 (Streptococcus thermophilus)—used for organisms with specific PAM requirements; HiFi Cas9 was developed as an alternative to Cas9 to create an enzyme that maintained potent on-target editing activity but had reduced off-target editing activity; Dead Cas9 (dCas9), a catalytically inactive form of Cas9. Used for gene regulation and visualization, as it can bind to DNA without cutting it; Cas12 (Cpf1), alternative to Cas9, derived from Francisella novicida (FnCpf1) and Acidaminococcus (AsCpf1), creates staggered (sticky) ends rather than blunt ends; Cas12a—recognizes a T-rich PAM, useful for AT-rich genomes; Cas12b—smaller Cas12 variant, suitable for viral delivery systems; Cas12f (Cpf1 Mini), a smaller variant useful for gene-editing applications with size constraints; AsCas12a Ultra is an enhanced variant of the original AsCas12a, a CRISPR-associated protein from the Cas12a family (formerly known as Cpf1) derived from Acidaminococcus species; MAD7/EURECA-V is a CRISPR-associated protein that belongs to the Cas12 family (a Type V CRISPR system). Developed by the company Inscripta, manufactured by Aldevron; Cas13, Targets RNA instead of DNA, useful for RNA interference and detection; Cas13a (formerly C2c2)—cleaves RNA and has been used in diagnostics; Cas13b—different RNA cleavage specificity and applications in gene silencing; Cas13d—smaller version of Cas13, enabling delivery via compact vectors; Cas3, Known for its processive degradation of DNA; Csfl, Type III (RNA-Targeting): Csm and Cmr Complexes, Type III CRISPR systems target RNA with Csm and Cmr protein complexes, Useful for viral RNA degradation in bacterial immunity.

CRISPR guide RNA (gRNA) directs the Cas9 enzyme to a specific location in the genome where it needs to make a cut. The gRNA is designed to match a target DNA sequence, ensuring the CRISPR-Cas9 system edits only the intended site. The guide RNA is made up of two main parts: CRISPR RNA (crRNA) which is a sequence of about 20 nucleotides that is complementary to the target DNA sequence. Its primary function is to guide the Cas9 protein to the exact location in the genome where the DNA cut should be made; and Trans-activating CRISPR RNA (tracrRNA) which aids in forming a stable complex with the Cas9 enzyme. It's necessary for the activation of the Cas9 protein, enabling it to perform its function as molecular scissors.

A CRISPR eukaryotic expression cassette typically consists of several elements that together allow the CRISPR system to function efficiently within eukaryotic cells. These elements include the necessary components for gene editing, such as the Cas protein (usually Cas9) and the guide RNA (gRNA), such as the sgRNA as disclosed herein. In certain embodiments as disclosed herein, additional components for a eukaryotic CRISPR expression cassette may include a promoter for Cas Protein Expression, such as CMV (Cytomegalovirus) promoter, EF1α (Elongation factor-la) promoter, Ubiquitin C (UbC) promoter, or Tissue-specific promoters for targeted Cas9 expression, e.g., neuron-specific promoters like Synapsin (Syn) or liver-specific like Albumin promoter. In certain embodiments as disclosed herein, additional components for a eukaryotic CRISPR expression cassette may include Cas Protein Coding Sequence, such as HiFi Cas, or SpCas9, Cpf1/Cas12a, SaCas9, or other Cas9 variants. In certain embodiments as disclosed herein, additional components for a eukaryotic CRISPR expression cassette may include Nuclear Localization Signal (NLS) to ensure proper transport of the Cas9 protein into the nucleus of the eukaryotic cell. In certain embodiments as disclosed herein, additional components for a eukaryotic CRISPR expression cassette may include a Promoter for gRNA Expression, such as a U6 promoter, an H1 promoter, or a Tissue-specific Pol II promoters. In certain embodiments as disclosed herein, additional components for a eukaryotic CRISPR expression cassette may include a Guide RNA (gRNA) Expression Unit, such as gRNA scaffold, or Multiplexing gRNAs. In certain embodiments as disclosed herein, additional components for a eukaryotic CRISPR expression cassette may include a Polyadenylation Signal (pA), or a Selectable Marker such as Antibiotic resistance genes, or fluorescent markers. In certain embodiments as disclosed herein the expression cassette may include Viral Vector Elements, such as Lentiviral vectors, AAV (Adeno-associated virus) vectors, or Self-inactivating (SIN) elements. In certain embodiments as disclosed herein the expression cassette may include Inducible Systems, such as Tet-On/Tet-Off systems or CRISPRa/i systems, or Insulator Sequences such as cHS4 insulators.

An NLS (Nuclear Localization Signal) is a short peptide sequence that will guide a protein to enter the nucleus of a cell, and can be added to proteins, for example, Cas9, to help them enter the nucleus of a eukaryotic cell. Since gene-editing processes like CRISPR-Cas9 target DNA, which is located in the cell's nucleus, it's essential that Cas proteins efficiently reach this compartment. The Nuclear Localization Signal (NLS) is a specific sequence of amino acids that is recognized by the cell's transport machinery. This sequence acts like a “tag” that signals the cell to transport the Cas protein into the nucleus. The NLS binds to nuclear import proteins, which then facilitate the passage of the Cas protein through nuclear pores, channels that regulate movement between the cytoplasm and the nucleus. By attaching an NLS to Cas proteins, scientists ensure that these proteins reach the nucleus quickly and efficiently, enabling precise and effective gene editing within the target DNA.

There are several effective strategies for introducing the sgRNAs and/or CRISPR components (like plasmids, ribonucleoprotein complexes, or mRNA) as disclosed herein as disclosed herein into target cells. Exemplary embodiments as disclosed herein include Viral Vectors, such as Adeno-Associated Virus (AAV) which are widely used for CRISPR delivery because they are generally safe, induce minimal immune response, and have been approved in some gene therapy applications. However, their small packaging capacity (around 4.7 kb) limits the size of CRISPR systems they can carry, so they work best for smaller Cas proteins (like Cas9 variants or Cas12a); Lentivirus and Retrovirus: Lentiviral vectors have a larger capacity than AAV and can integrate the CRISPR components into the host genome, allowing for stable, long-term expression. However, this integration can cause insertional mutagenesis; Adenovirus: Adenovirus vectors can carry larger payloads, including the standard SpCas9 and multiple gRNAs. They are non-integrating, but they can induce stronger immune responses, which may limit their use in some settings.

Additional methods for introducing the sgRNAs and/or CRISPR proteins as disclosed herein into target cells includes, for example, Lipid Nanoparticles (LNPs) which are commonly used for delivering RNA-based therapies, including mRNA for Cas proteins and gRNA complexes. They are a non-viral delivery method that is scalable and relatively low-risk, with minimal immune response and no genomic integration. LNPs are currently used in clinical applications and are effective for delivery in vivo, especially in the liver and other tissues with good blood flow; Electroporation, which involves applying an electrical field to create temporary pores in the cell membrane, allowing the sgRNAs and/or CRISPR components (like plasmids, ribonucleoprotein complexes, or mRNA) as disclosed herein to enter the cell. It is especially effective for cell lines, primary cells, and immune cells such as T-cells. This method is efficient but can be harsh on sensitive cells, leading to higher cell mortality. Additional methods for introducing the sgRNAs and/or CRISPR components (like plasmids, ribonucleoprotein complexes, or mRNA) as disclosed herein into target cells includes, for example, Ribonucleoprotein (RNP) Complexes which involves directly delivering the Cas9 protein pre-complexed with guide RNA, such as the sgRNA as disclosed herein into cells, usually via electroporation or lipid-based transfection. This approach has advantages: it minimizes the risk of off-target effects, reduces immune response, and is transient, avoiding genomic integration. Lipid-Based Transfection Agents (lipofection) uses lipid-based agents to encapsulate CRISPR plasmids or RNP complexes and facilitate their uptake by cells. This is straightforward and widely used for cell lines, but its efficiency can vary across cell types and is generally less effective for primary or difficult-to-transfect cells.

Other methods for introducing the sgRNAs and/or CRISPR components (for example, plasmids, ribonucleoprotein complexes, or mRNA) as disclosed herein into target cells includes, for example, physical methods such as Microinjection, which directly injects the sgRNAs and/or CRISPR components (for example, plasmids, ribonucleoprotein complexes, or mRNA) as disclosed herein into cells, typically used in single-cell embryos or zygotes for generating transgenic animals. This is a precise but labor-intensive approach; Nanoneedles and Microfluidics: Emerging physical methods like nanoneedles or microfluidic devices can introduce the sgRNAs and/or CRISPR components (for example, plasmids, ribonucleoprotein complexes, or mRNA) as disclosed herein with minimal damage to cells. They're promising for in vitro applications and high-throughput settings but are still being developed. Exosome-Mediated Delivery, which can be engineered to carry the sgRNAs and/or CRISPR components (for example, plasmids, ribonucleoprotein complexes, or mRNA) as disclosed herein and target them to specific cells. This is a promising, non-viral, cell-derived delivery method that may allow for targeted delivery with minimal immune response.

Next Generation Sequencing (NGS) allows rapid and high-throughput sequencing of DNA and RNA. Unlike earlier methods such as Sanger sequencing, which sequences one DNA fragment at a time, NGS enables the simultaneous sequencing of millions of DNA fragments, making it much faster, cheaper, and more efficient. In NGS, a DNA or RNA from the sample is extracted and fragmented into smaller pieces. These fragments are then attached to short synthetic DNA sequences called adapters, which are needed for binding to the sequencing platform. The DNA fragments with adapters are amplified (copied many times) to create a “library” of DNA fragments. This increases the amount of DNA available for sequencing. Most NGS platforms, like Illumina, use a method called “sequencing by synthesis.” Each fragment is attached to a solid surface and copied in place. Fluorescently labeled nucleotides (A, T, C, and G) are added one by one. As they bind to the complementary strand, the machine detects the fluorescent signal, allowing the sequence of bases to be read. The massive amount of sequencing data is analyzed using bioinformatics tools. The overlapping DNA fragments are assembled back into their original sequence by aligning them to a reference genome or constructing new genomes (de novo sequencing). NGS allows for High Throughput, since millions to billions of DNA fragments can be sequenced in parallel, producing vast amounts of data, is cost-effective, and can sequence entire genomes or large sets of genes in days, making it much faster than older sequencing methods.

Our previous filings in this and related areas include the following: U.S. patent application Ser. No. 16/536,256, filed Mar. 22, 2021, and entitled “NOVEL MUTATIONS THAT ENHANCE THE DNA CLEAVAGE ACTIVITY OF Acidaminococcus SP. CPF1” to Liyang Zhang et al.; U.S. patent application Ser. No. 17/857,262, filed Jul. 5, 2022, and entitled “NOVEL MUTATIONS IN Streptococcus pyogenes CAS9 DISCOVERED BY BROAD SCANNING MUTAGENESIS DEMONSTRATE ENHANCEMENT OF DNA CLEAVAGE ACTIVITY” to Nathaniel Hunter Roberts et al.; U.S. patent application Ser. No. 17/952,252, filed Sep. 24, 2022, and entitled “UBIQUITIN VARIANTS WITH IMPROVED AFFINITY FOR 53BP1” to Christopher Anthony Vakulskas et al.; U.S. patent application Ser. No. 17/988,275, filed Nov. 16, 2022, and entitled “DESIGN OF TWO-PART GUIDE RNAS FOR CRISPRa APPLICATIONS” to Javier Alejandro Gomez Vargas et al.; and U.S. patent application Ser. No. 18/450,149, filed Aug. 15, 2023, and entitled “A UBIQUITIN VARIANT WITH HIGH AFFINITY FOR BINDING 53BP1 REDUCES THE AMOUNT OF AAV NEEDED TO ACHIEVE HIGH RATES OF HDR” to Steve Ehren Glenn et al.

CM1/Ubv-A can be used to improve the rate of homology directed repair following the inducement of a double strand break through delivery of a 53BP1-binding ubiquitin variant into cells. Improving the rate of HDR allows for increased rates of successful genome editing using the CRISPR/Cas9 system or other targeted nucleases in conjunction with supplying a repair template to direct precise genome editing events. This work explores the use of Ubv-A in conjunction with MMEJ inhibition and the triple combination of MMEJ inhibition, NHEJ inhibition, and end protection inhibition for maximizing HDR rates for experiments involving HDR repair of targeted double strand breaks for the purposes of genome editing.

Recent publications have shown that the combination of NHEJ inhibition and MMEJ inhibition can improve rates of HDR while reducing the frequency of unintended effects such as translocations¹⁶. This work advances upon that work by showing that Ubv-A can be used in conjunction with both MMEJ and NHEJ inhibition to further boost the level of HDR that can be achieved. It also demonstrates that use of MMEJ inhibition in conjunction with 53BP1 inhibition provides additional benefit to HDR over 53BP1 inhibition alone. This is also the first demonstration that Ubv-A facilitates HDR with staggered breaks induced by AsCas12a and ErCas12a. It also demonstrates that Ubv-A can be delivered as either protein or mRNA alongside either Cas9 protein or Cas9 mRNA to promote HDR.

The present disclosure pertains to methods for improving HDR rates in the context of HDR-based genome editing by inhibiting end protection mediated by 53BP1 in combination with inhibition of MMEJ, NHEJ, or MMEJ and NHEJ. Inhibition of 53BP1 has been shown to increase rates of end resection and promote MMEJ and HDR over NHEJ^{50, 65}.

The compounds and methods as disclosed herein provide that a 53BP1 binding ubiquitin variant, such as Ubv-A, can be used in conjunction with an NHEJ inhibitor, Alt-R HDR enhancer V2 (V2), or an MMEJ inhibitor, ART558, or both to further improve HDR rates. We tested whether our 53BP1 inhibitor, Ubv-A, can be used in conjunction with MMEJ inhibition through the use of a DNA polymerase theta inhibitor (ART558) to further boost rates of HDR. We also tested if the combination of 53BP1 inhibition via Ubv-A and NHEJ inhibition via V2 could be supplemented with MMEJ inhibition via ART558 to further increase rates of HDR. We found that while MMEJ inhibition alone usually had little effect on rates of HDR, combining MMEJ inhibition with Ubv-A or V2 often had a positive synergistic effect on HDR rates. Further, combining NHEJ inhibition, end protection inhibition, and MMEJ inhibition further increased rates of HDR and overall purity of repair outcome. We find that inhibiting combinations of end protection+MMEJ, end protection+NHEJ, and end protection+NHEJ+MMEJ are useful mechanisms for HDR-based CRISPR genome editing.

### Applications

In a first aspect, a method for improving the rate of double-strand break (DSB) repair by homology directed repair (HDR) at a DNA target site in a cell by a targeting DNA endonuclease is provided. The method includes a step of introducing into the cell genome editing agents and recombination enhancing agents. The genome editing agents include the targeting DNA endonuclease and a donor DNA. The recombination enhancing agents include a first component, a second component, and optionally a third component. The first component inhibits 53BP1-mediated DNA end protection. The second component inhibits microhomology-mediated end joining (MMEJ). The third component inhibits non-homologous end joining (NHEJ). The rate of double-strand break (DSB) repair by homology directed repair (HDR) at the DNA target site by the targeting DNA endonuclease is improved.

In a first respect, the first component includes a Ubv-A molecule, the second component includes a MMEJ inhibitor ART558 molecule, and the third component includes a NHEJ inhibitor Alt-R HDR enhancer V2 molecule. In a second respect, the Ubv-A molecule is selected from a Ubv-A polypeptide or a nucleic acid encoding a Ubv-A polypeptide. In a third respect, the nucleic acid encoding a Ubv-A polypeptide is selected from a mRNA encoding a Ubv-A polypeptide, or a DNA encoding a Ubv-A polypeptide. According to the first aspect and the foregoing respects, the targeting DNA endonuclease includes a Clustered Regularly Interspaced Short Palindromic Repeat (CRISPR) endonuclease. In this regard, the Clustered Regularly Interspaced Short Palindromic Repeat (CRISPR) endonuclease is selected from AsCas12a Ultra or SpCas9. In another embodiment, the Clustered Regularly Interspaced Short Palindromic Repeat (CRISPR) endonuclease is supplied as a Ribonucleoprotein complex comprising a CRISPR polypeptide and a suitable guide RNA or as a mixture of nucleic acids encoding CRISPR polypeptide and a suitable guide RNA.

In a second aspect, a method for improving the rate of double-strand break (DSB) repair by homology directed repair (HDR) at a DNA target site in a cell by a DNA endonuclease is provided. The method includes the step of introducing into the cell genome editing agents and recombination enhancing agents. The genome editing agents include the targeting DNA endonuclease and a donor DNA. The recombination enhancing agents include a first component that inhibits 53BP1-mediated DNA end protection and a second component that inhibits non-homologous end joining (NHEJ). The rate of double-strand break (DSB) repair by homology directed repair (HDR) at a DNA target site by a DNA endonuclease is improved.

In a first respect, the first component includes a Ubv-A molecule and the second component includes a NHEJ inhibitor Alt-R HDR enhancer V2 molecule. In a second respect, the Ubv-A molecule is selected from a Ubv-A polypeptide or a nucleic acid encoding a Ubv-A polypeptide. In a third respect, the nucleic acid encoding a Ubv-A polypeptide is selected from a mRNA encoding a Ubv-A polypeptide, or a DNA encoding a Ubv-A polypeptide. In the second aspect and the foregoing respects, the targeting DNA endonuclease includes a Clustered Regularly Interspaced Short Palindromic Repeat (CRISPR) endonuclease. In third respect, the Clustered Regularly Interspaced Short Palindromic Repeat (CRISPR) endonuclease is selected from AsCas12a Ultra or SpCas9. In another embodiment, the Clustered Regularly Interspaced Short Palindromic Repeat (CRISPR) endonuclease is supplied as a Ribonucleoprotein complex comprising a CRISPR polypeptide and a suitable guide RNA or as a mixture of nucleic acids encoding CRISPR polypeptide and a suitable guide RNA.

In a third aspect, a kit for use in improving the rate of double-strand break (DSB) repair by homology directed repair (HDR) at a DNA target site by a targeting DNA endonuclease is provided. The kit includes a recombination enhancing agent that improves the rate of double-strand break repair by homology directed repair at the DNA target site by the targeting DNA endonuclease. The recombination enhancing agent includes a member selected from the group consisting of an inhibitor of 53BP1-mediated DNA end protection; an inhibitor of microhomology-mediated end joining (MMEJ); an inhibitor of non-homologous end joining (NHEJ), or a combination thereof.

In a first respect, the recombination enhancing agent includes the inhibitor of 53BP1-mediated DNA end protection and a member selected from the group consisting of the inhibitor of microhomology-mediated end joining (MMEJ) and the inhibitor of non-homologous end joining (NHEJ). In a second respect, the recombination enhancing agent includes the inhibitor of 53BP1-mediated DNA end protection and the inhibitor of microhomology-mediated end joining (MMEJ). In a third respect, the recombination enhancing agent includes the inhibitor of 53BP1-mediated DNA end protection and the inhibitor of non-homologous end joining (NHEJ). In the third aspect and the foregoing respects, the inhibitor of 53BP1-mediated DNA end protection includes a Ubv-A molecule, the inhibitor of microhomology-mediated end joining (MMEJ) includes the MMEJ inhibitor ART558 molecule, and the inhibitor of non-homologous end joining (NHEJ) includes the NHEJ inhibitor Alt-R HDR enhancer V2 molecule. In another respect, the recombination enhancing agent which inhibits 53BP1-mediated DNA end protection is a 53BP1 binding ubiquitin variant selected from the group consisting of a Ubv-A polypeptide; an mRNA encoding a Ubv-A polypeptide; and a nucleic acid encoding a Ubv-A polypeptide. In the third aspect and the foregoing respects, the kit also includes the targeting DNA endonuclease. In this regard, the targeting DNA endonuclease includes a Clustered Regularly Interspaced Short Palindromic Repeat (CRISPR) endonuclease. In another respect, the Clustered Regularly Interspaced Short Palindromic Repeat (CRISPR) endonuclease is selected from AsCas12a Ultra or SpCas9. In another embodiment, the Clustered Regularly Interspaced Short Palindromic Repeat (CRISPR) endonuclease is supplied as a Ribonucleoprotein complex including a CRISPR polypeptide and a suitable guide RNA or as a mixture of nucleic acids encoding CRISPR polypeptide and a suitable guide RNA. In the third aspect and the foregoing respects, the kit also includes instructions of the method of the first and/or second aspects.

### Methods

Small insert SpCas9, AsCas12a, and EURECA-V (a ErCas12a variant) testing

For HDR using ssDNA templates in HEK293 cells genome editing was mediated via IDT Alt-R Cas9 ribonucleoprotein, IDT Alt-R AsCas12a Ultra, or EURECA-V (a ErCas12a variant) (RNP) complexes delivered by Lonza nucleofection in concert with single-stranded oligodeoxynucleotide (ssODN) HDR repair templates. The specific repair event was the insertion of a 6-nt EcoRI sequence (5′-GAATTC-3′) at either the Cas9, AsCas12a, or ErCas12a cut site. The sgRNAs for Cas9 and crRNAs for AsCas12a corresponding to the protospacer sequences listed in Table 2 were ordered from IDT (Table 2). The HDR donor templates for Cas9 editing contain 40-nt homology arms (HA) flanking a 6 nt EcoR1 cut site insert. The HDR donor templates for AsCas12a were similar except for slightly different homology arm lengths (40 nt 5′ HA and 45 nt 3′ HA)⁶⁶. The HDR donor sequences listed in Table 3 were ordered from IDT as Alt-R HDR donor oligos (Table 3). The repair templates are homologous to the non-targeting strand of dsDNA, where targeting/non-targeting is defined with respect to the guide RNA sequence and the presence of the NGG/TTTV PAM sequence identifying the non-target strand. The RNPs were generated by complexing Cas9, Cas12a, or ErCas12a at a 1:1.2 molar ratio of protein to guide to give the indicated final concentration for each figure where the final concentration of Cas9 RNP refers to the concentration of Cas9 in the final cells, protein, RNA, and DNA mix. The Ubv-A protein was used at 25 μM concentration for all experiments. The ART558 DNA polymerase theta inhibitor was purchased from MedChemExpress (Cat. No. HY-141520). Cas9/Cas12a RNP, donor, and Ubv protein was delivered into HEK293 cells using the Lonza 96-well Shuttle with the pulse code DS-150. Measurement of HDR by NGS was performed according to the rhAmpSeq™ CRISPR library preparation protocol available on IDT's website using the primers listed in Table 4 for PCR 1 and standard i5 and i7 indexing primers for PCR2 (Table 4). This technology makes use of blocked primers containing a single ribonucleotide residue which are activated by cleavage by RNase H2 as part of the PCR amplification during library generation^{67, 68}. NGS libraries were then sequenced on a MiSeq® platform (Illumina®) using 2×150 bp PE sequencing and processed through an internal IDT processing pipeline. For mRNA experiments, Ubv-A mRNA was produced by in vitro transcription (Table 5).

### Chimeric Antigen Receptor (CAR) Knock-In at T Cell Receptor Alpha Constant (TRAC) Locus

HEK293 cells were harvested and resuspended in SF Cell Line Nucleofector Solution (Lonza, V4SC-2096) at a concentration of approximately 16,500 cells/μl and 18 μl of this cell suspension was used for each nucleofection. Nucleofection mixes also contained 2 μM RNP or 1 μg of Cas9 mRNA with 4.8 μM sgRNA, 25 μM Ubv-A peptide or 25 nM Ubv-A mRNA, and 1 μg of an Alt-R HDR donor block as indicated. The Alt-R HDR donor block contained 500 base-pair homology arms and was designed to insert a CAR sequence into the TRAC locus. Cells were nucleofected with a 4D-Nucleofector 96-well Unit (Lonza, AAF-10035) using the pulse code DS-150, seeded into a 96-well plate (approximately 37,500 cells/well), and cultured in DMEM containing DMSO, 1 μM Alt-R HDR enhancer V2, 10 μM ART558, or both 1 μM Alt-R HDR enhancer V2 and 10 μM ART558 for approximately 21 hours. Approximately 76 hours after nucleofections, genomic DNA was isolated from cells with QuickExtract DNA Extraction Solution (BioSearch Technologies, SS000035-D2). To quantify knock-in percentages, genomic DNA flanking the sgRNA target site was amplified by PCR, PCR products were separated on a Fragment Analyzer (Agilent Technologies), and the percentage of edited and unedited alleles was calculated using PROSize 3.0 software.

### EXAMPLES

### Example 1. Inhibition of End Protection Through Use of Ubv-A in Combination with Inhibition of NHEJ, MMEJ, or NHEJ Plus MMEJ Improves Rates of HDR

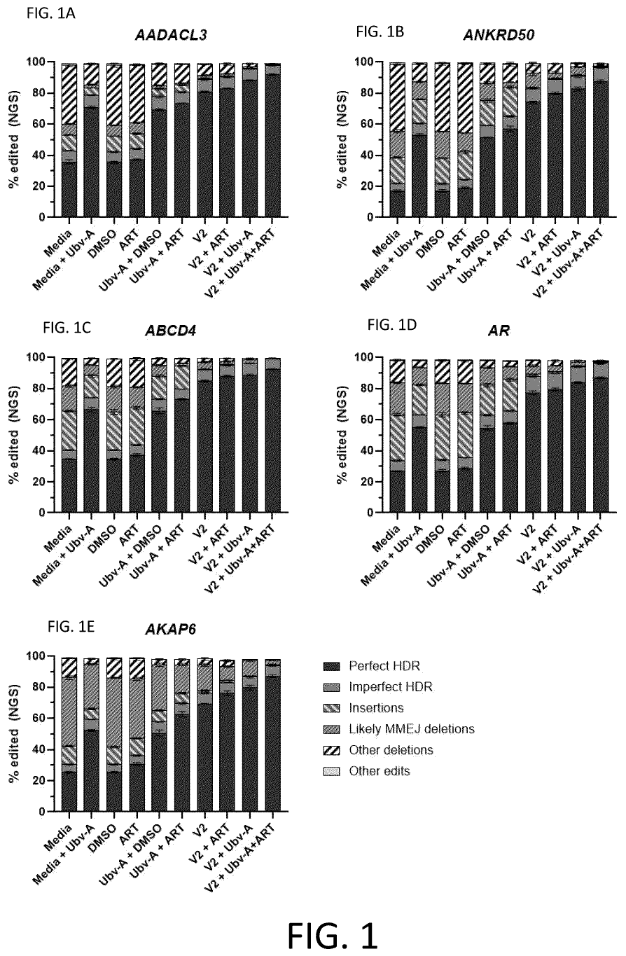

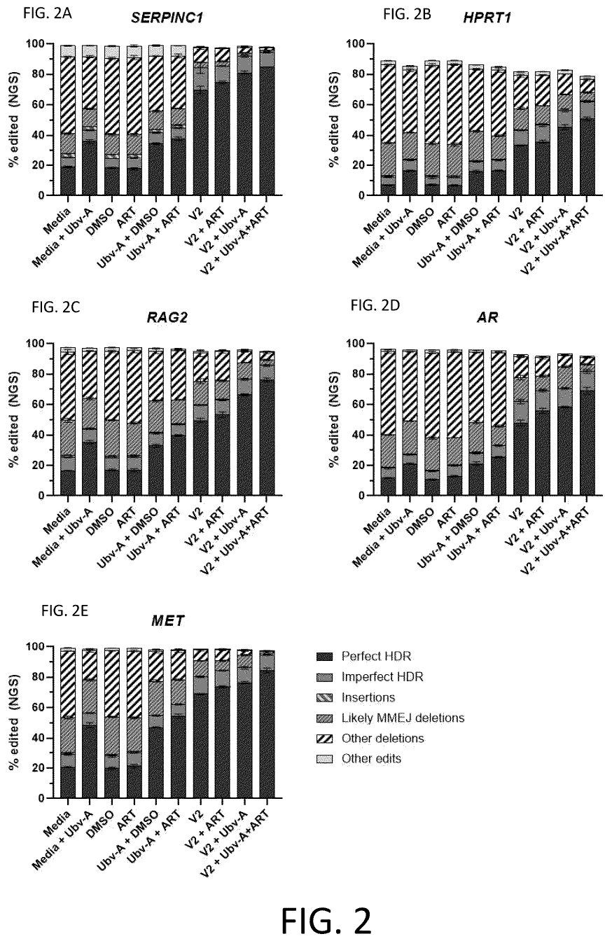

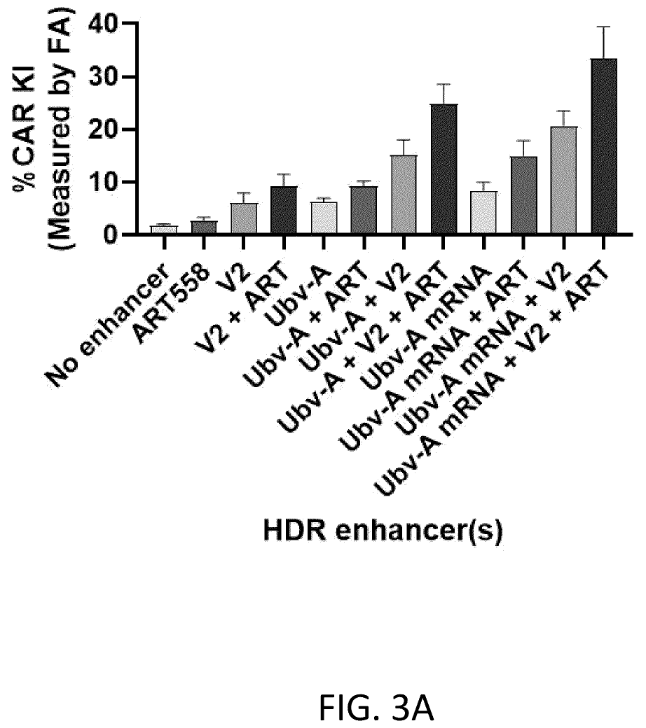

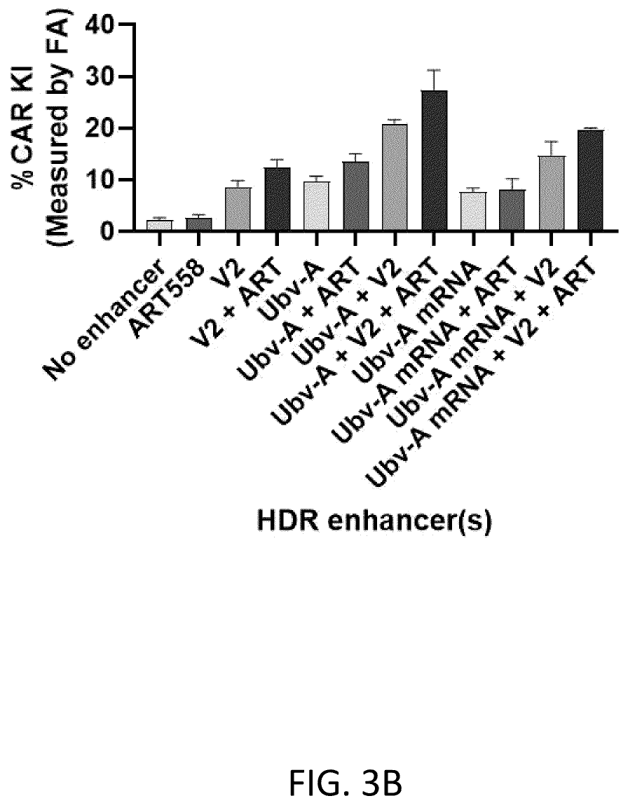

In previous filings we demonstrated that use of Ubv-A to inhibit 53BP1 can be combined with inhibition of NHEJ using Alt-R HDR enhancer V2 (V2) to further boost HDR. Recent publications have shown that inhibition of MMEJ through inhibition of DNA polymerase theta using small molecule inhibitors increases the rate of HDR and reduces undesired edits^{16,27}. Given that we see a shift towards MMEJ repair upon treatment of cells with Ubv-A when no donor template is supplied, we hypothesized that use of an MMEJ inhibitor may help promote HDR when combined with Ubv-A. To test this, we delivered 2 μM Cas9 RNP alongside 2 μM ssDNA for introduction of a 6 bp insert with and without 25 μM Ubv-A into HEK293 cells. Cells were then plated into media, media with DMSO, media with 10 μM ART558 (MedchemExpress), media with 1 μM V2, or media with 10 μM ART558 and 1 μM V2. All small molecule containing media had had the same concentration of DMSO as the control DMSO media (0.29%). After 24 hours, media was removed from all wells and fresh media was added. Genomic DNA was isolated approximately 48 hours after delivery and editing was analyzed by NGS. The results are shown in FIG. 1. Use of ART558 alone provided a minimal boost in HDR. The site for which there was the largest increase in HDR with ART558 alone was AKAP6 which had the highest level of likely MMEJ repair events in the media only condition. When ART558 was combined with Ubv-A there was a notable increase in rates of HDR alongside a decrease in likely MMEJ events while other non-HDR repair events were largely unaffected. This is also observed for use of ART558 in combination with V2 though the effect is less pronounced. The largest increase in HDR was observed with use of Ubv-A in combination with both V2 and ART558. The same combination of enhancers was also tested using A.s. Cas12a Ultra RNP instead of Cas9. The results are shown in FIG. 2. Despite the notably different repair profile, Ubv-A was similarly beneficial across all the sites tested and a similar pattern of additional benefit using the triple combination of enhancers was observed, demonstrating that the combinations are beneficial for HDR repair of both staggered and blunt breaks. We also tested these combinations of enhancers for the ability to facilitate large insertions. To do this, we attempted to insert a CAR coding sequence at the TRAC locus using a long dsDNA donor. In addition to the previously tested combinations of enhancers, we also tested delivery of Ubv-A as mRNA as well as delivery of Cas9 as mRNA. The results of this testing are shown in FIG. 3. Used alone, ART558 had a minimal effect on HDR while the use of V2, Ubv-A mRNA, or Ubv-A protein resulted in a dramatic improvement in HDR rates. Use of Ubv-A and V2 together appeared to be approximately additive while adding ART558 to either V2 or Ubv-A resulted in a synergistic increase in HDR. The combination of all three enhancers by far provided the largest increase to HDR, demonstrating the benefit of adding end protection inhibition by Ubv-A to the recently published combination of MMEJ and NHEJ inhibition. These results indicate that combining inhibition of NHEJ, MMEJ, and end protection is a useful approach for maximizing rates of HDR.

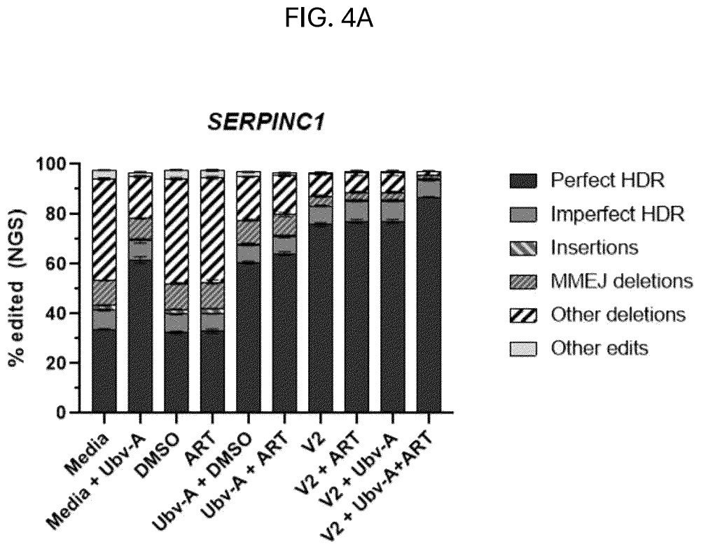

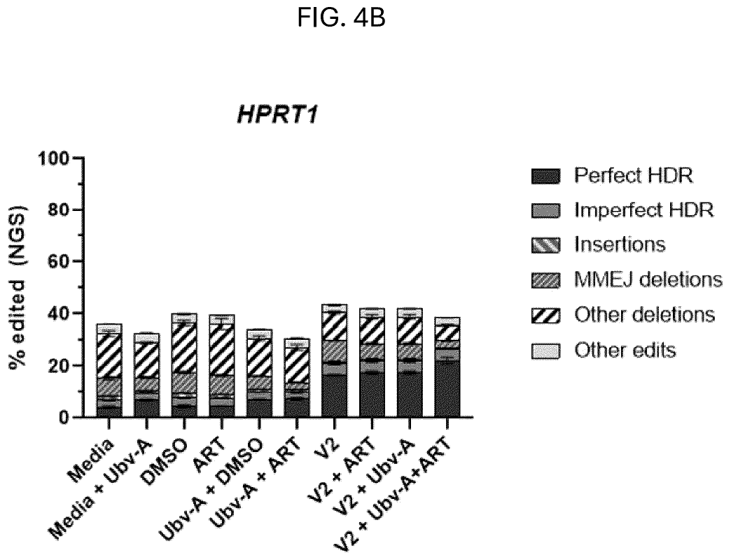

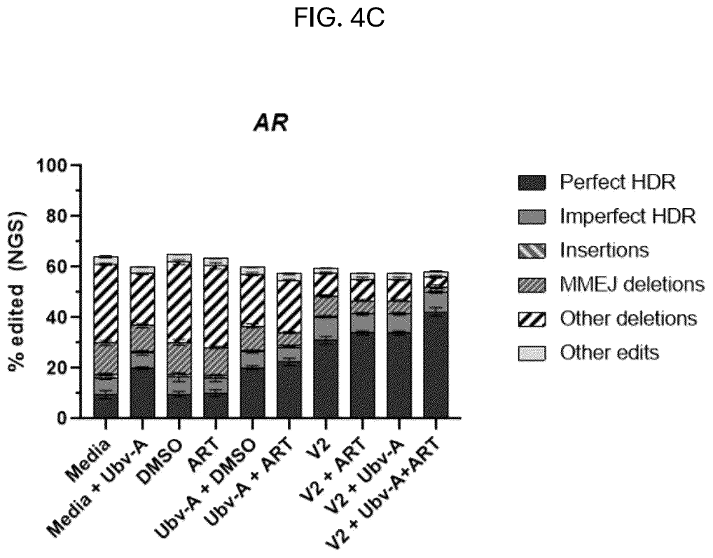

Inhibition of NHEJ, MMEJ, and 53BP1 leads to high levels of HDR at EURECA-V induced DSBs. The results of the testing are shown in FIG. 4A to FIG. 4C. HEK293 cells were treated with EURECA-V (3 μM, Aldevron) targeting either the SERPINC1 site (FIG. 4A), the HPRT1 site (FIG. 4B), or the AR site (FIG. 4C) in the genome alongside ssDNA donor template (2 μM) with or without Ubv-A protein (25 μM). Cells were then plated in media containing no DMSO, DMSO control, ART558 (ART, 10 μM, MedChemExpress HY-141520), Alt-R HDR enhancer V2 (1 μM, IDT), or ART+V2 for 24 hours before exchanging for fresh media. The graphs show the percent of each repair event under each condition measured by NGS with error bars indicating SD from three replicates. Likely MMEJ deletions are determined based on the presence of microhomology of two nucleotides or greater at the ends of the deletion. These data demonstrate that Ubv-A can be used in combination with EURECA-V (a ErCas12a variant). These data demonstrate that Ubv-A is effective at boosting HDR to a similar degree whether a cut is blunt, as with SpCas9, or staggered, as with AsCas12a or ErCas12a. (Table 6).

### Example 2. Tables of Amino Acid and Nucleic Acid Sequences Used

TABLE 1
Amino acid and DNA sequences

| SEQ ID | | Protein | |
|---|---|---|---|
| NO:ª | Name | Sequence | DNA sequence |
| 1; 2 | Ubv-A | MLIFVRMLT | ATGTTGATTTTCGTACGCAT |
| | (previously | GKMIELEVE | GTTGACTGGAAAGATGATCG |
| | called CM1) | PSDTIENVK | AGTTGGAAGTGGAGCCTTCC |
| | | AKIQDHEGI | GATACTATCGAGAATGTTAA |
| | | PPDQQRLAF | GGCCAAAATCCAAGATCATG |
| | | QGKSLEDGR | AAGGGATTCCTCCAGATCAA |
| | | TLSDYNILK | CAACGCCTTGCTTTTCAAGG |
| | | DPKKMPLLR | GAAGAGCCTGGAGGACGGTC |
| | | LR | GCACACTGTCTGACTATAAC |
| | | | ATTCTTAAAGATCCTAAAAA |
| | | | GATGCCACTGCTGCGCTTGC |
| | | | GT |

ªThe first SEQ ID NO. refers to the amino acid sequence while the second SEQ ID NO. refers to the nucleic acid sequence.

TABLE 2
Guide designs

| SEQ ID NO: | Name | For use with protein | Protospacer Sequences |
|---|---|---|---|
| 4 | SERPINC1 | AsCas12a Ultra and | GAGGTCACAAAACCCAGTAGG |
| | crRNA | EURECA-V | |
| 5 | HPRT1 crRNA | AsCas12a Ultra and | AACAGACTGATGGTTCCCATT |
| | | EURECA-V | |
| 6 | RAG2 crRNA | AsCas12a Ultra | CTGGTGGATTTTGAATTTGGG |
| 7 | AR crRNA | AsCas12a Ultra and | CGTGGTTGCTCCCGCAAGTTT |
| | | EURECA-V | |
| 8 | MET crRNA | AsCas12a Ultra | TTTACTTCTTGACGGTCCAAA |
| 9 | AADACL3 | Cas9 | GGCATCGTGTACTACCACGG |
| | sgRNA | | |
| 10 | ANKRD50 | Cas9 | TCGGAAGGCATATATCGTCA |
| | sgRNA | | |
| 11 | ABCD4 | Cas9 | CTGGTAAGTGTAGTAGACGA |
| | sgRNA | | |
| 12 | AR sgRNA | Cas9 | CTGGGACGCAACCTCTCTCG |
| 13 | AKAP6 sgRNA | Cas9 | ACGCCAATGGCAACTACACT |
| 14 | TRAC sgRNA | Cas9 | GAGAATCAAAATCGGTGAAT |

TABLE 3
donor DNA sequences

| SEQ ID NO: | Name | Experiment | DNA sequence |
|---|---|---|---|
| 15 | SERPINC1 | AsCas12a and | GAGGTCATTCCTGTGAGTCCTTTGGAGGTCA |
| | donor | EURECA-V | CAAAACCCAGAATTCGTAGGGGCAGGCAAG |
| | | testing | GGGAAAGCTCACCCCTCTTACCTTTTTCCA |
| 16 | HPRT1 donor | AsCas12a and | CTCCTTCCAGCACCTCATAATTTGAACAGACT |
| | | EURECA-V | GATGGTTCGAATTCCCATTAGTCACATAAAGC |
| | | testing | TGTAGTCTAGTACAGACGTCCTTAGAA |
| 17 | RAG2 donor | AsCas12a testing | GCTGACTGCCTGCCCTGTGTTTTCCTGGTGGA |
| | | | TTTTGAATGAATTCTTGGGTGTGCTACATCAT |
| | | | ACATTCTTCCAGAACTTCAGGATGGGC |
| 18 | AR donor | AsCas12a and | TTGAGGCTGTCAGAGCGCTTTTTGCGTGGTT |
| | | EURECA-V testing | GCTCCCGCAGAATTCAGTTTCCTTCTCTGGAG |
| | | | CTTCCCGCAGGTGGGCAGCTAGCTGCAG |
| 19 | MET donor | AsCas12a testing | ATGCCTTTGAAAGCAACAATTTTATTTACTTCT |
| | | | TGACGGTGAATTCCCAAAGGGAAACTCTAGA |
| | | | TGCTCAGACTTTTCACACAAGAATAAT |
| 20 | AADACL3 | Cas9 testing | CATCCACCTGCACCCTGAAGCCTGGCATCGT |
| | donor | | GTACTACCAGAATTCCGGTGGCGGGGGCGTC |
| | | | ATGGGGAGTTTGAGTAAGAACCAT |
| 21 | ANKRD50 | Cas9 testing | CGAAAAATAAGTTTAGATGACCTTCGGAAGG |
| | | | CATATATCGGAATTCTCAAGGATGTTCAGCA |
| | donor | | GTACATTCTTCATCGTTTAGATCA |
| 22 | ABCD4 donor | Cas9 testing | GGGAGGCTTCACCTTTGGAAGCACTGGTAAG |
| | | | TGTAGTAGAGAATTCCGAGGGTGAACGGGG |
| | | | AGATGATGAGCTTGCTGGCCATGCT |
| 23 | AR donor | Cas9 testing | GGCGGCCACGGCGGCTCCAGGCTCTGGGAC |
| | | | GCAACCTCTCGAATTCTCGGGGTGGCACTCC |
| | | | AGGGCCGACTGCGGCTGTGAAGGTT |
| 24 | AKAP6 donor | Cas9 testing | GCGCATTCTGCAAGGTCTGCAGGACGCCAAT |
| | | | GGCAACTACGAATTCACTAGGCAGACGGACA |
| | | | TTCTGCAAGCTTTCTCTGAAGAGA |
| 25 | TRAC donor | Cas9 testing | GTCGTACCGACTGGTAGATGACAGCAAACCC |
| | | | CACTCCAGATTCCAAGATGTACAGTTTGCTTT |
| | | | GCTGGGCCTTTTTCCCATGCCTGCCTTTACTCT |
| | | | GCCAGAGTTATATTGCTGGGGTTTTGAAGAA |
| | | | GATCCTATTAAATAAAAGAATAAGCAGTATT |
| | | | ATTAAGTAGCCCTGCATTTCAGGTTTCCTTGA |
| | | | GTGGCAGGCCAGGCCTGGCCGTGAACGTTCA |
| | | | CTGAAATCATGGCCTCTTGGCCAAGATTGAT |
| | | | AGCTTGTGCCTGTCCCTGAGTCCCAGTCCATC |
| | | | ACGAGCAGCTGGTTTCTAAGATGCTATTTCCC |
| | | | GTATAAAGCATGAGACCGTGACTTGCCAGCC |
| | | | CCACAGAGCCCCGCCCTTGTCCATCACTGGCA |
| | | | TCTGGACTCCAGCCTGGGTTGGGGCAAAGAG |
| | | | GGAAATGAGATCATGTCCTAACCCTGATCCTC |
| | | | TTGTCCCACAGATATCCAGAACCCTGACCCTG |
| | | | CCGTGTACCAGCTGAGAGACTCTAAATCCAG |
| | | | TGACAAGTCTGTCTGCCTATTCACCGCCACCA |
| | | | ACTTCTCTCTTTTGAAGCAGGCCGGAGATGT |
| | | | GGAAGAAAATCCTGGGCCCATGGCTCTTCCT |
| | | | GTGACTGCCCTTCTGCTGCCCCTGGCTCTCCT |
| | | | GCTCCATGCTGCCCGGCCAGACATCCAGATG |
| | | | ACACAGACAACCAGCAGCCTCAGCGCCAGCC |
| | | | TGGGGGACAGAGTGACCATTAGCTGCCGGG |
| | | | CCTCTCAGGACATCAGCAAATACCTGAACTG |
| | | | GTACCAGCAGAAACCAGATGGCACTGTCAAG |
| | | | CTGCTGATTTACCACACCTCCAGGCTCCACAG |
| | | | CGGCGTGCCCAGTCGCTTCAGCGGCAGTGGG |
| | | | AGCGGGACAGATTATTCCCTCACAATCTCCAA |
| | | | CCTGGAGCAGGAAGATATTGCCACATACTTC |
| | | | TGCCAGCAAGGCAACACCCTGCCATACACAT |
| | | | TTGGAGGCGGCACCAAATTGGAGATCACCG |
| | | | GCGGTGGTGGATCTGGAGGAGGGGGCAGC |
| | | | GGAGGTGGCGGCTCTGAGGTGAAACTGCAG |
| | | | GAGAGTGGCCCTGGCCTGGTGGCTCCCAGCC |
| | | | AGAGCCTTTCTGTCACCTGCACCGTGTCTGGG |
| | | | GTGTCCCTGCCTGACTATGGAGTCTCCTGGAT |
| | | | CCGGCAGCCTCCAAGAAAAGGACTGGAATG |
| | | | GCTGGGCGTCATCTGGGGAAGTGAGACCAC |
| | | | CTACTATAATTCAGCCCTCAAGTCCCGGCTCA |
| | | | CCATCATTAAGGACAACTCCAAATCCCAGGT |
| | | | GTTCCTGAAGATGAATTCTCTCCAGACTGATG |
| | | | ACACAGCCATCTACTACTGTGCCAAGCATTAT |
| | | | TATTACGGCGGGTCCTATGCCATGGACTACT |
| | | | GGGGCCAGGGGACCAGTGTCACTGTTTCTTC |
| | | | TGAAAGCAAGTATGGCCCCCCATGCCCTCCCT |
| | | | GCCCCGGACAGCCGCGGGAGCCCCAGGTCT |
| | | | ACACCCTCCCACCCTCCAGAGATGAGCTGACC |
| | | | AAGAACCAGGTTTCACTGACATGCCTGGTGA |
| | | | AGGGCTTCTACCCCTCTGACATTGCTGTGGA |
| | | | GTGGGAGAGCAATGGGCAGCCAGAGAACAA |
| | | | CTACAAGACCACACCTCCTGTCCTGGACAGC |
| | | | GACGGCTCCTTCTTCCTCTACAGTAAGCTCAC |
| | | | TGTGGACAAGAGCCGCTGGCAGCAGGGCAA |
| | | | TGTCTTCTCCTGCAGCGTGATGCACGAGGCC |
| | | | CTGCACAATGCCTATACCCAGAAGTCACTCTC |
| | | | TCTGAGCCCTGGAAAGAAGGATCCCAAGTTT |
| | | | TGGGTCCTCGTGGTGGTCGGGGGTGTGCTG |
| | | | GCCTGCTACAGCTTGCTGGTCACAGTGGCCTT |
| | | | CATCATCTTCTGGGTGCGCTCCAAGAGGAGC |
| | | | CGGCTGCTTCACAGTGATTACATGAACATGA |
| | | | CCCCCAGGAGGCCAGGACCCACCAGGAAGC |
| | | | ACTACCAGCCCTACGCTCCCCCGCGGGACTTT |
| | | | GCTGCTTACCGCAGCAGGGTCAAATTTTCTA |
| | | | GATCTGCAGATGCGCCGGCCTATCAACAAGG |
| | | | CCAGAACCAGCTCTATAACGAGCTCAATCTA |
| | | | GGACGAAGAGAGGAGTACGATGTTTTGGAC |
| | | | AAGAGACGTGGCCGGGACCCTGAGATGGGG |
| | | | GGAAAGCCGAGAAGGAAGAACCCTCAGGAA |
| | | | GGCCTGTACAATGAACTGCAGAAAGATAAGA |
| | | | TGGCGGAGGCCTACAGTGAGATTGGGATGA |
| | | | AAGGCGAGCGCCGGAGGGGCAAGGGGCAC |
| | | | GATGGCCTTTACCAGGGTCTCAGTACAGCCA |
| | | | CCAAGGACACCTACGACGCCCTTCACATGCA |
| | | | GGCCCTGCCCCCTCGCTAATAAGAATTCTAAC |
| | | | TAGAGCTCGCTGATCAGCCTCGACTGTGCCTT |
| | | | CTAGTTGCCAGCCATCTGTTGTTTGCCCCTCC |
| | | | CCCGTGCCTTCCTTGACCCTGGAAGGTGCCAC |
| | | | TCCCACTGTCCTTTCCTAATAAAATGAGGAAA |
| | | | TTGCATCGCATTGTCTGAGTAGGTGTCATTCT |
| | | | ATTCTGGGGGGTGGGGTGGGGCAGGACAGC |
| | | | AAGGGGGAGGATTGGGAAGAGAATAGCAG |
| | | | GCATGCTGGGGAGCGGCCGCGTTAAATTTTG |
| | | | ATTCTCAAACAAATGTGTCACAAAGTAAGGA |
| | | | TTCTGATGTGTATATCACAGACAAAACTGTGC |
| | | | TAGACATGAGGTCTATGGACTTCAAGAGCAA |
| | | | CAGTGCTGTGGCCTGGAGCAACAAATCTGAC |
| | | | TTTGCATGTGCAAACGCCTTCAACAACAGCAT |
| | | | TATTCCAGAAGACACCTTCTTCCCCAGCCCAG |
| | | | GTAAGGGCAGCTTTGGTGCCTTCGCAGGCTG |
| | | | TTTCCTTGCTTCAGGAATGGCCAGGTTCTGCC |
| | | | CAGAGCTCTGGTCAATGATGTCTAAAACTCCT |
| | | | CTGATTGGTGGTCTCGGCCTTATCCATTGCCA |
| | | | CCAAAACCCTCTTTTTACTAAGAAACAGTGAG |
| | | | CCTTGTTCTGGCAGTCCAGAGAATGACACGG |
| | | | GAAAAAAGCAGATGAAGAGAAGGTGGCAGG |
| | | | AGAGGGCACGTGGCCCAGCCTCAGTCTCTCC |
| | | | AACTGAGTTCCTGCCTGCCTGCCTTTGCTCAG |
| | | | ACTGTTTGCCCCTTACTGCTCGATCCGAAAAC |
| | | | GGGCGTATAGTCGAGACC |

TABLE 4
Primer sequences

| SEQ ID NO: | Gene | Assay | Primer | Primer Sequenceª |
|---|---|---|---|---|
| 26 | SERPINCI | NGS for Cas12a testing | Forward | AGGTCTTTGACTGTAACTACCAGrGGAGA |
| 27 | | | Reverse | GAAGATTAGCGGCCATGTATTCrCAATG |
| 28 | HPRTI | NGS for Cas12a testing | Forward | CTCACTCATTTTTCAAAACACGCrATAAA |
| 29 | | | Reverse | ACTATTTCAGCCAACAAGAAGTIGTCAC |
| 30 | RAG2 | NGS for Cas12a testing | Forward | TAGATGGTGTCATTTTTGGCAATArGAGAC |
| 31 | | | Reverse | GAAAAGTATGGGTGTTCTCTTTGrGAGGA |
| 32 | AR | NGS for Cas12a testing | Forward | AGACAGACTGTGAGCCTAGrCAGGG |
| 33 | | | Reverse | AGAGTTCAACAGGCTGTGATrGATGC |
| 34 | MET | NGS for Cas12a testing | Forward | GACCAGTCCTACATTGATGTTTTACrCTGAG |
| 35 | | | Reverse | GTAGGAATGCAATCCAGAGTTTATrGGAAC |
| 36 | AADACL3 | NGS for Cas9 testing | Forward | ATTTCCGCTTTGGGACAATCrCCTGT |
| 37 | | | Reverse | GAAGATGCCCATTCTTCCAArGTTAT |
| 38 | ANKRD50 | NGS for Cas9 testing | Forward | CATCTCTGCAGTTTCTTTTGTGAGrGTGTT |
| 39 | | | Reverse | GTTGACATTCTCTTTGAAGATATGGTrACTTC |
| 40 | ABCD4 | NGS for Cas9 testing | Forward | TTCTCTTGGACCTTGACCTGrGGAGA |
| 41 | | | Reverse | TGTGTCATTGGCTCCAGGrGACCA |
| 42 | AR | NGS for Cas9 testing | Forward | GTTCTCCCCAAGCCCATrCGTAG |
| 43 | | | Reverse | AGCTGAGTCATCCTCGTCrCGGAG |
| 44 | AKAP6 | NGS for Cas9 testing | Forward | TCTTACTCTGTCAACGTGATAGTrGGACA |
| 45 | | | Reverse | CATTGCTTAACTTCAAGGAAAACArCTCAC |
| 46 | TRAC | PCR for FA analysis | Forward | TTTCTGTTGGTGCTGATATTGCGAGAGAGCA |
| | | of CAR | | ATCTCCTGGTAATG |
| 47 | | insertion at TRAC | Reverse | ACTTGCCTGTCGCTCTATCTTCCTGACTTAGT |
| | | | | GAGCTGGGAAAG |

^{a}Primer sequences that include an “r” designation refer to an RNA residue having a ribose moiety.

TABLE 5

| SEQ | | |
|---|---|---|
| ID | | |
| NO: | Name | mRNA sequence |
| 3 | Ubv-A | GGGAGACCCAAGCUGGCUAGCGUUUAAACGG |
| | mRNA | GCCCUCUAGACUCGAGCGGCCGCcaccAUG |
| | | CUGAUCUUCGUGAGAAUGCUGACCGGCAAG |
| | | AUGAUCGAACUGGAAGUGGAACCCAGCGAC |
| | | ACCAUCGAGAACGUGAAGGCCAAAAUCCAG |
| | | GACCACGAGGGCAUCCCUCCUGACCAGCAG |
| | | AGACUGGCCUUUCAGGGAAAGUCCCUGGAA |
| | | GAUGGAAGAACCCUGAGCGACUACAACAUC |
| | | CUGAAGGACCCUAAGAAGAUGCCACUGCUG |
| | | AGACUGAGAUGAUCAGCCUCGACUGUGCCU |
| | | UCUAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAAAAAAAAAAAAAAAAAAAAAAAAAAAA |
| | | AAAA |

TABLE 6
CRISPR DNA endonuclease amino acid and
nucleic acid sequences

| SEQ | | |
|---|---|---|
| ID | | |
| NO: | Name | Sequence |
| 48 | Cas9 Protein | MDKKYSIGLDIGTNSVGWAVITDEYKVPSK |
| | | KFKVLGNTDRHSIKKNLIGALLFDSGETAE |
| | | ATRLKRTARRRYTRRKNRICYLQEIFSNEM |
| | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTVA |
| | | YSVLVVAKVEKGKSKKLKSVKELLGITIME |
| | | RSSFEKNPIDFLEAKGYKEVKKDLIIKLPK |
| | | YSLFELENGRKRMLASAGELQKGNELALPS |
| | | KYVNFLYLASHYEKLKGSPEDNEQKQLFVE |
| | | QHKHYLDEIIEQISEFSKRVILADANLDKV |
| | | LSAYNKHRDKPIREQAENIIHLFTLTNLGA |
| | | PAAFKYFDTTIDRKRYTSTKEVLDATLIHQ |
| | | SITGLYETRIDLSQLGGD |
| 49 | Cas9 DNA | ATGGACAAAAAGTACTCTATTGGCCTGGAT |
| | | ATCGGGACCAACAGCGTCGGGTGGGCTGTT |
| | | ATCACCGACGAGTATAAAGTACCTTCGAAA |
| | | AAGTTCAAAGTGCTGGGCAACACCGATCGC |
| | | CATTCAATCAAAAAGAACTTGATTGGTGCG |
| | | CTGTTGTTTGACTCCGGGGAAACCGCCGAG |
| | | GCGACTCGCCTTAAACGTACAGCACGTCGC |
| | | CGGTACACTCGGCGTAAGAATCGCATTTGC |
| | | TATTTGCAGGAAATCTTTAGCAACGAGATG |
| | | GCAAAAGTCGATGACTCGTTTTTCCACCGC |
| | | CTCGAGGAAAGCTTTCTGGTGGAGGAAGAC |
| | | AAAAAGCATGAGCGTCACCCGATCTTCGGC |
| | | AACATTGTCGATGAAGTAGCGTATCATGAA |
| | | AAATACCCAACCATTTACCACTTACGCAAA |
| | | AAGCTGGTGGACAGCACTGACAAAGCTGAT |
| | | TTGCGCCTTATCTATTTAGCCCTGGCACAT |
| | | ATGATTAAGTTTCGTGGTCACTTCCTGATC |
| | | GAAGGAGACTTAAATCCCGACAACAGTGAT |
| | | GTTGATAAATTGTTTATTCAGCTTGTCCAA |
| | | ACTTACAATCAACTGTTCGAGGAAAACCCG |
| | | ATCAATGCCTCCGGTGTGGATGCAAAAGCC |
| | | ATTTTAAGTGCACGCCTTAGCAAGTCCCGT |
| | | CGCTTAGAAAACCTTATCGCGCAGCTGCCC |
| | | GGCGAGAAAAAGAATGGTTTGTTTGGGAAC |
| | | CTTATTGCCTTGAGCTTAGGCCTCACCCCG |
| | | AATTTCAAAAGTAATTTCGATCTTGCAGAA |
| | | GACGCCAAATTACAACTGTCGAAGGATACT |
| | | TATGATGACGATCTCGATAATCTGTTAGCG |
| | | CAGATTGGTGACCAATACGCCGATCTTTTT |
| | | CTGGCGGCTAAAAATCTGAGCGACGCCATC |
| | | TTGCTTTCGGATATTCTCCGCGTTAACACC |
| | | GAAATCACGAAAGCGCCTCTTAGTGCCAGC |
| | | ATGATTAAACGTTATGATGAACACCACCAG |
| | | GACCTGACCTTACTCAAAGCGTTGGTTCGC |
| | | CAGCAACTGCCAGAGAAGTACAAAGAAATC |
| | | TTCTTTGATCAGTCAAAGAATGGTTATGCC |
| | | GGCTATATTGACGGGGGTGCAAGCCAAGAG |
| | | GAATTCTACAAATTTATCAAGCCTATTCTG |
| | | GAGAAAATGGATGGCACCGAAGAGTTATTG |
| | | GTGAAGCTTAACCGTGAAGACCTCCTGCGG |
| | | AAACAGCGCACATTCGATAATGGTTCGATC |
| | | CCACACCAAATCCATTTGGGGGAGTTACAC |
| | | GCTATTTTGCGTCGCCAGGAAGACTTTTAC |
| | | CCTTTCCTGAAGGATAACCGGGAGAAAATT |
| | | GAGAAGATCCTTACCTTTCGTATTCCGTAT |
| | | TACGTAGGCCCCTTAGCACGGGGTAATAGC |
| | | CGTTTCGCGTGGATGACACGGAAGTCGGAA |
| | | GAGACGATCACCCCGTGGAACTTCGAAGAG |
| | | GTAGTCGACAAGGGCGCATCAGCGCAGTCT |
| | | TTTATTGAACGTATGACGAATTTCGATAAA |
| | | AACTTGCCCAATGAGAAGGTGCTTCCGAAA |
| | | CATTCCTTGTTATATGAATATTTTACAGTT |
| | | TACAACGAGCTGACCAAGGTTAAATACGTG |
| | | ACGGAAGGAATGCGCAAGCCCGCTTTTCTT |
| | | AGCGGTGAGCAAAAAAAGGCGATCGTCGAC |
| | | CTGTTATTCAAAACGAATCGTAAGGTGACT |
| | | GTAAAGCAACTCAAAGAAGATTACTTCAAA |
| | | AAGATTGAGTGCTTCGACAGCGTCGAAATC |
| | | TCTGGGGTAGAGGATCGGTTTAACGCAAGT |
| | | TTAGGTACCTACCATGACCTGCTTAAAATC |
| | | ATTAAGGATAAAGACTTCTTAGATAATGAA |
| | | GAGAACGAAGATATTCTCGAGGACATCGTC |
| | | TTGACGTTAACCTTATTTGAGGATCGTGAA |
| | | ATGATTGAGGAACGCCTCAAAACTTATGCC |
| | | CACCTGTTCGACGATAAGGTGATGAAGCAG |
| | | CTGAAACGTCGGCGCTACACAGGATGGGGC |
| | | CGCTTGAGTCGCAAACTTATTAACGGAATC |
| | | CGTGACAAGCAATCCGGCAAAACGATTCTG |
| | | GATTTCTTGAAGTCGGACGGATTTGCTAAT |
| | | CGCAACTTCATGCAGTTGATCCATGATGAC |
| | | TCCCTGACTTTTAAAGAGGATATTCAAAAG |
| | | GCGCAGGTTAGTGGTCAAGGCGACAGCTTA |
| | | CACGAACACATCGCAAATTTGGCTGGTTCG |
| | | CCGGCCATTAAAAAGGGGATCCTCCAGACC |
| | | GTGAAAGTTGTAGATGAGCTTGTTAAGGTC |
| | | ATGGGTCGTCATAAGCCCGAAAACATCGTG |
| | | ATTGAAATGGCGCGGGAGAATCAAACGACC |
| | | CAGAAAGGACAAAAGAATAGCCGTGAACGG |
| | | ATGAAGCGGATCGAGGAAGGCATTAAAGAG |
| | | CTGGGGTCTCAAATCTTGAAGGAACACCCT |
| | | GTGGAGAACACTCAGCTCCAAAATGAAAAA |
| | | CTTTACCTGTACTATTTGCAGAACGGACGC |
| | | GATATGTACGTGGACCAAGAGTTGGATATT |
| | | AATCGGCTGAGTGACTACGACGTTGATCAT |
| | | ATCGTCCCGCAGAGCTTCCTCAAAGACGAT |
| | | TCTATTGACAATAAGGTACTGACGCGCTCT |
| | | GATAAAAACCGTGGTAAGTCGGACAACGTG |
| | | CCCTCCGAAGAGGTTGTGAAAAAGATGAAA |
| | | AATTATTGGCGCCAGCTTTTAAACGCGAAG |
| | | CTGATCACACAACGTAAATTCGATAATTTG |
| | | ACCAAGGCTGAACGGGGTGGCCTGAGCGAG |
| | | TTAGATAAGGCAGGATTTATTAAACGCCAG |
| | | TTAGTGGAGACTCGTCAAATCACCAAACAT |
| | | GTCGCGCAGATTTTGGACAGCCGGATGAAC |
| | | ACCAAGTACGATGAAAATGACAAACTGATC |
| | | CGTGAGGTGAAAGTCATTACTCTGAAGTCC |
| | | AAATTAGTTAGTGATTTCCGGAAGGACTTT |
| | | CAATTCTACAAAGTCCGTGAAATTAATAAC |
| | | TATCATCACGCACATGACGCGTACCTGAAT |
| | | GCAGTGGTTGGGACCGCCCTTATCAAGAAA |
| | | TATCCTAAGCTGGAGTCGGAGTTTGTCTAT |
| | | GGCGACTATAAGGTATACGATGTTCGCAAA |
| | | ATGATTGCGAAATCTGAGCAGGAGATCGGT |
| | | AAGGCAACCGCAAAATATTTCTTTTACTCA |
| | | AACATTATGAATTTCTTTAAGACAGAAATC |
| | | ACTCTGGCCAACGGGGAGATTCGCAAACGT |
| | | CCGTTGATCGAAACAAACGGCGAGACTGGC |
| | | GAAATTGTTTGGGACAAAGGGCGTGATTTC |
| | | GCGACGGTGCGCAAGGTACTGAGCATGCCT |
| | | CAAGTCAATATTGTTAAGAAAACCGAAGTG |
| | | CAGACGGGCGGGTTTTCCAAGGAAAGCATC |
| | | TTACCCAAACGTAATTCAGATAAACTTATT |
| | | GCACGCAAAAAGGACTGGGATCCGAAAAAG |
| | | TATGGAGGCTTCGACAGTCCAACCGTAGCC |
| | | TACTCTGTTCTCGTTGTAGCGAAAGTAGAA |
| | | AAGGGTAAATCCAAGAAACTGAAATCTGTC |
| | | AAGGAGTTGCTTGGAATCACCATTATGGAG |
| | | CGTAGCTCCTTCGAGAAGAACCCGATTGAC |
| | | TTTCTGGAAGCCAAAGGATATAAAGAGGTC |
| | | AAGAAAGATCTTATCATTAAGCTGCCTAAG |
| | | TATTCACTCTTCGAGCTGGAAAATGGTCGT |
| | | AAACGCATGCTCGCTTCTGCCGGCGAGTTG |
| | | CAGAAGGGCAATGAATTAGCACTTCCATCA |
| | | AAGTACGTTAACTTCCTGTATTTGGCCAGC |
| | | CATTACGAGAAACTGAAGGGGTCTCCAGAG |
| | | GACAACGAACAGAAACAATTATTTGTAGAG |
| | | CAGCACAAGCATTATCTTGATGAAATCATT |
| | | GAGCAAATTTCCGAATTCAGTAAACGCGTA |
| | | ATCCTGGCCGATGCAAACCTCGACAAGGTG |
| | | CTGAGCGCTTACAATAAGCATCGCGACAAA |
| | | CCTATCCGTGAGCAGGCTGAAAATATCATT |
| | | CACCTGTTCACATTAACGAACCTGGGCGCT |
| | | CCGGCCGCTTTTAAATATTTCGACACGACA |
| | | ATCGACCGTAAGCGCTATACCAGTACGAAA |
| | | GAAGTGTTGGATGCGACCCTTATTCACCAG |
| | | TCAATTACAGGATTATATGAGACCCGTATC |
| | | GACCTTAGCCAATTAGGTGGGGAT |
| 50 | AsCas12a | MTQFEGFTNLYQVSKTLRFELIPQGKTLKH |
| | Ultra | IQEQGFIEEDKARNDHYProteinKELKPI |
| | | IDRIYKTYADQCLQLVQLDWENLSAAIDSY |
| | | RKEKTEETRNALIEEQATYRNAIHDYFIGR |
| | | TDNLTDAINKRHAEIYKGLFKAELFNGKVL |
| | | KQLGTVTTTEHENALLRSFDKFTTYFSGFY |
| | | ENRKNVFSAEDISTAIPHRIVQDNFPKFKE |
| | | NCHIFTRLITAVPSLREHFENVKKAIGIFV |
| | | STSIEEVFSFPFYNQLLTQTQIDLYNQLLG |
| | | GISREAGTEKIKGLNEVLNLAIQKNDETAH |
| | | IIASLPHRFIPLFKQILSDRNTLSFILEEF |
| | | KSDEEVIQSFCKYKTLLRNENVLETAEALF |
| | | NELNSIDLTHIFISHKKLETISSALCDHWD |
| | | TLRNALYERRISELTGKITKSAKEKVQRSL |
| | | KHEDINLQEIISAAGKELSEAFKOKTSEIL |
| | | SHAHAALDQPLPTTLKKQEEKEILKSQLDS |
| | | LLGLYHLLDWFAVDESNEVDPEFSARLTGI |
| | | KLEMEPSLSFYNKARNYATKKPYSVEKFKL |
| | | NFQRPTLASGWDVNKEKNNGAILFVKNGLY |
| | | YLGIMPKQKGRYKALSFEPTEKTSEGFDKM |
| | | YYDYFPDAAKMIPKCSTQLKAVTAHFQTHT |
| | | TPILLSNNFIEPLEITKEIYDLNNPEKEPK |
| | | KFQTAYAKKTGDQKGYREALCKWIDFTRDF |
| | | LSKYTKTTSIDLSSLRPSSQYKDLGEYYAE |
| | | LNPLLYHISFQRIAEKEIMDAVETGKLYLF |
| | | QIYNKDFAKGHHGKPNLHTLYWTGLFSPEN |
| | | LAKTSIKLNGQAELFYRPKSRMKRMAHRLG |
| | | EKMLNKKLKDQKTPIPDTLYQELYDYVNHR |
| | | LSHDLSDEARALLPNVITKEVSHEIIKDRR |
| | | FTSDKFLFHVPITLNYQAANSPSKFNQRVN |
| | | AYLKEHPETPIIGIDRGERNLIYITVIDST |
| | | GKILEQRSLNTIQQFDYQKKLDNREKERVA |
| | | ARQAWSVVGTIKDLKQGYLSQVIHEIVDLM |
| | | IHYQAVVVLENLNFGFKSKRTGIAEKAVYQ |
| | | QFEKMLIDKLNCLVLKDYPAEKVGGVLNPY |
| | | QLTDQFTSFAKMGTQSGFLFYVPAPYTSKI |
| | | DPLTGFVDPFVWKTIKNHESRKHFLEGFDF |
| | | LHYDVKTGDFILHFKMNRNLSFQRGLPGFM |
| | | PAWDIVFEKNETQFDAKGTPFIAGKRIVPV |
| | | IENHRFTGRYRDLYPANELIALLEEKGIVE |
| | | RDGSNILPKLLENDDSHAIDTMVALIRSVL |
| | | QMRNSNAATGEDYINSPVRDLNGVCFDSRF |
| | | QNPEWPMDADANGAYHIALKGQLLLNHLKE |
| | | SKDLKLQNGISNQDWLAYIQELRN |
| 51 | AsCas12a | atgACCCAGTTTGAAGGTTTCACCAATCTG |
| | Ultra | TATCAGGTTAGCAAAADNACCCTGCGTTTT |
| | | GAACTGATTCCGCAGGGTAAAACCCTGAAA |
| | | CATATTCAAGAACAGGGCTTCATCGAAGAG |
| | | GATAAAGCACGTAACGATCACTACAAAGAA |
| | | CTGAAACCGATTATCGACCGCATCTATAAA |
| | | ACCTATGCAGATCAGTGTCTGCAGCTGGTT |
| | | CAGCTGGATTGGGAAAATCTGAGCGCAGCA |
| | | ATTGATAGTTATCGCAAAGAAAAAACCGAA |
| | | GAAACCCGTAATGCACTGATTGAAGAACAG |
| | | GCAACCTATCGTAATGCCATCCATGATTAT |
| | | TTCATTGGTCGTACCGATAATCTGACCGAT |
| | | GCAATTAACAAACGTCACGCCGAAATCTAT |
| | | AAAGGCCTGTTTAAAGCCGAACTGTTTAAT |
| | | GGCAAAGTTCTGAAACAGCTGGGCACCGTT |
| | | ACCACCACCGAACATGAAAATGCACTGCTG |
| | | CGTAGCTTTGATAAATTCACCACCTATTTC |
| | | AGCGGCTTTTATGAGAATCGCAAAAACGTG |
| | | TTTAGCGCAGAAGATATTAGCACCGCAATT |
| | | CCGCATCGTATTGTGCAGGATAATTTCCCG |
| | | AAATTCAAAGAGAACTGCCACATTTTTACC |
| | | CGTCTGATTACCGCAGTTCCGAGCCTGCGT |
| | | GAACATTTTGAAAACGTTAAAAAAGCCATC |
| | | GGCATCTTTGTTAGCACCAGCATTGAAGAA |
| | | GTTTTTAGCTTCCCGTTTTACAATCAGCTG |
| | | CTGACCCAGACCCAGATTGATCTGTATAAC |
| | | CAACTGCTGGGTGGTATTAGCCGTGAAGCA |
| | | GGCACCGAAAAAATCAAAGGTCTGAATGAA |
| | | GTGCTGAATCTGGCCATTCAGAAAAATGAT |
| | | GAAACCGCACATATTATTGCAAGCCTGCCG |
| | | CATCGTTTTATTCCGCTGTTCAAACAAATT |
| | | CTGAGCGATCGTAATACCCTGAGCTTTATT |
| | | CTGGAAGAATTCAAATCCGATGAAGAGGTG |
| | | ATTCAGAGCTTTTGCAAATACAAAACGCTG |
| | | CTGCGCAATGAAAATGTTCTGGAAACTGCC |
| | | GAAGCACTGTTTAACGAACTGAATAGCATT |
| | | GATCTGACCCACATCTTTATCAGCCACAAA |
| | | AAACTGGAAACCATTTCAAGCGCACTGTGT |
| | | GATCATTGGGATACCCTGCGTAATGCCCTG |
| | | TATGAACGTCGTATTAGCGAACTGACCGGT |
| | | AAAATTACCAAAAGCGCGAAAGAAAAAGTT |
| | | CAGCGCAGTCTGAAACATGAGGATATTAAT |
| | | CTGCAAGAGATTATTAGCGCAGCCGGTAAA |
| | | GAACTGTCAGAAGCATTTAAACAGAAAACC |
| | | AGCGAAATTCTGTCACATGCACATGCAGCA |
| | | CTGGATCAGCCGCTGCCGACCACCCTGAAA |
| | | AAACAAGAAGAAAAAGAAATCCTGAAAAGC |
| | | CAGCTGGATAGCCTGCTGGGTCTGTATCAT |
| | | CTGCTGGACTGGTTTGCAGTTGATGAAAGC |
| | | AATGAAGTTGATCCGGAATTTAGCGCACGT |
| | | CTGACCGGCATTAAACTGGAAATGGAACCG |
| | | AGCCTGAGCTTTTATAACAAAGCCCGTAAT |
| | | TATGCCACCAAAAAACCGTATAGCGTCGAA |
| | | AAATTCAAACTGAACTTTCAGCGTCCGACC |
| | | CTGGCAAGCGGTTGGGATGTTAATAAAGAA |
| | | AAAAACAACGGTGCCATCCTGTTCGTGAAA |
| | | AATGGCCTGTATTATCTGGGTATTATGCCG |
| | | AAACAGAAAGGTCGTTATAAAGCGCTGAGC |
| | | TTTGAACCGACGGAAAAAACCAGTGAAGGT |
| | | TTTGATAAAATGTACTACGACTATTTTCCG |
| | | GATGCAGCCAAAATGATTCCGAAATGTAGC |
| | | ACCCAGCTGAAAGCAGTTACCGCACATTTT |
| | | CAGACCCATACCACCCCGATTCTGCTGAGC |
| | | AATAACTTTATTGAACCGCTGGAAATCACC |
| | | AAAGAGATCTACGATCTGAATAACCCGGAA |
| | | AAAGAGCCGAAAAAATTCCAGACCGCATAT |
| | | GCAAAAAAAACCGGTGATCAGAAAGGTTAT |
| | | CGTGAAGCGCTGTGTAAATGGATTGATTTC |
| | | ACCCGTGATTTTCTGAGCAAATACACCAAA |
| | | ACCACCAGTATCGATCTGAGCAGCCTGCGT |
| | | CCGAGCAGCCAGTATAAAGATCTGGGCGAA |
| | | TATTATGCAGAACTGAATCCGCTGCTGTAT |
| | | CATATTAGCTTTCAGCGTATTGCCGAGAAA |
| | | GAAATCATGGACGCAGTTGAAACCGGTAAA |
| | | CTGTACCTGTTCCAGATCTACAATAAAGAT |
| | | TTTGCCAAAGGCCATCATGGCAAACCGAAT |
| | | CTGCATACCCTGTATTGGACCGGTCTGTTT |
| | | AGCCCTGAAAATCTGGCAAAAACCTCGATT |
| | | AAACTGAATGGTCAGGCGGAACTGTTTTAT |
| | | CGTCCGAAAAGCCGTATGAAACGTATGGCA |
| | | CATCGTCTGGGTGAAAAAATGCTGAACAAA |
| | | AAACTGAAAGACCAGAAAACCCCGATCCCG |
| | | GATACACTGTATCAAGAACTGTATGATTAT |
| | | GTGAACCATCGTCTGAGCCATGATCTGAGT |
| | | GATGAAGCACGTGCCCTGCTGCCGAATGTT |
| | | ATTACCAAAGAAGTTAGCCACGAGATCATT |
| | | AAAGATCGTCGTTTTACCAGCGACAAATTC |
| | | CTGTTTCATGTGCCGATTACCCTGAATTAT |
| | | CAGGCAGCAAATAGCCCGAGCAAATTTAAC |
| | | CAGCGTGTTAATGCATATCTGAAAGAACAT |
| | | CCAGAAACGCCGATTATTGGTATTGATCGT |
| | | GGTGAACGTAACCTGATTTATATCACCGTT |
| | | ATTGATAGCACCGGCAAAATCCTGGAACAG |
| | | CGTAGCCTGAATACCATTCAGCAGTTTGAT |
| | | TACCAGAAAAAACTGGATAATCGCGAGAAA |
| | | GAACGTGTTGCAGCACGTCAGGCATGGTCA |
| | | GTTGTTGGTACAATTAAAGACCTGAAACAG |
| | | GGTTATCTGAGCCAGGTTATTCATGAAATT |
| | | GTGGATCTGATGATTCACTATCAGGCCGTT |
| | | GTTGTGCTGGAAAACCTGAATTTTGGCTTT |
| | | AAAAGCAAACGTACCGGCATTGCAGAAAAA |
| | | GCAGTTTATCAGCAGTTCGAGAAAATGCTG |
| | | ATTGACAAACTGAATTGCCTGGTGCTGAAA |
| | | GATTATCCGGCTGAAAAAGTTGGTGGTGTT |
| | | CTGAATCCGTATCAGCTGACCGATCAGTTT |
| | | ACCAGCTTTGCAAAAATGGGCACCCAGAGC |
| | | GGATTTCTGTTTTATGTTCCGGCACCGTAT |
| | | ACGAGCAAAATTGATCCGCTGACCGGTTTT |
| | | GTTGATCCGTTTGTTTGGAAAACCATCAAA |
| | | AACCATGAAAGCCGCAAACATTTTCTGGAA |
| | | GGTTTCGATTTTCTGCATTACGACGTTAAA |
| | | ACGGGTGATTTCATCCTGCACTTTAAAATG |
| | | AATCGCAATCTGAGTTTTCAGCGTGGCCTG |
| | | CCTGGTTTTATGCCTGCATGGGATATTGTG |
| | | TTTGAGAAAAACGAAACACAGTTCGATGCA |
| | | AAAGGCACCCCGTTTATTGCAGGTAAACGT |
| | | ATTGTTCCGGTGATTGAAAATCATCGTTTC |
| | | ACCGGTCGTTATCGCGATCTGTATCCGGCA |
| | | AATGAACTGATCGCACTGCTGGAAGAGAAA |
| | | GGTATTGTTTTTCGTGATGGCTCAAACATT |
| | | CTGCCGAAACTGCTGGAAAATGATGATAGC |
| | | CATGCAATTGATACCATGGTTGCACTGATT |
| | | CGTAGCGTTCTGCAGATGCGTAATAGCAAT |
| | | GCAGCAACCGGTGAAGATTACATTAATAGT |
| | | CCGGTTCGTGATCTGAATGGTGTTTGTTTT |
| | | GATAGCCGTTTTCAGAATCCGGAATGGCCG |
| | | ATGGATGCAGATGCAAATGGTGCATATCAT |
| | | ATTGCACTGAAAGGACAGCTGCTGCTGAAC |
| | | CACCTGAAAGAAAGCAAAGATCTGAAACTG |
| | | CAAAACGGCATTAGCAATCAGGATTGGCTG |
| | | GCATATATCCAAGAACTGCGTAAC |
| SEQ | EURECA-V | MNNGTNNFQNFIGISSLQKTLRNALIPTETT |
| ID | | QQFIVKNGIIKEDELRGENRQILKDIMDDY |
| NO: | | YRGFISETLSSIDDIDWTSLFEKMEIQLKN |
| 73 | | GDNKDTLIKEQTEYRKAIHKKFANDDRFKN |
| | | MFSAKLISDILPEFVIHNNNYSASEKEEKT |
| | | QVIKLFSRFATSFKDYFKNRANCFSADDIS |
| | | SSSCHRIVNDNAEIFFSNALVYRRIVKSLS |
| | | NDDINKISGDMKDSLKEMSLEEIYSYEKYG |
| | | EFITQEGISFYNDICGKVNSFMNLYCQKNK |
| | | ENKNLYKLQKLHKQILCIADTSYEVPYKFE |
| | | SDEEVYQSVNGFLDNISSKHIVERLRKIGD |
| | | NYNGYNLDKIYIVSKFYESVSQKTYRDWET |
| | | INTALEIHYNNILPGNGKSKADKVKKAVKN |
| | | DLQKSITEINELVSNYKLCSDDNIKAETYI |
| | | HEISHILNNFEAQELKYNPEIHLVESELKA |
| | | SELKNVLDVIMNAFHWCSVFMTEELVDKDN |
| | | NFYAELEEIYDEIYPVISLYNLVRNYVTQK |
| | | PYSTKKIKLNFGIPTLADGWSKSKEYSNNA |
| | | IILMRDNLYYLGIFNAKNKPDKKIIEGNTS |
| | | ENKGDYKKMIYNLLPGPNKMIPKVFLSSKT |
| | | GVETYKPSAYILEGYKQNKHIKSSKDFDIT |
| | | FCHDLIDYFKNCIAIHPEWKNFGFDFSDTS |
| | | TYEDISGFYREVELQGYKIDWTYISEKDID |
| | | LLQEKGQLYLFQIYNKDFSKKSTGNDNLHT |
| | | MYLKNLFSEENLKDIVLKLNGEAEIFFRKS |
| | | SIKNPIIHKKGSILVNRTYEAEEKDQFGNI |
| | | QIVRKNIPENIYQELYKYFNDKSDKELSDE |
| | | AAKLKNVVGHHEAATNIVKDYRYTYDKYFL |
| | | HMPITINFKANKTGFINDRILQYIAKEKDL |
| | | HVIGIDRGERNLIYVSVIDTCGNIVEQKSF |
| | | NIVNGYDYQIKLKQQEGARQIARKEWKEIG |
| | | KIKEIKEGYLSLVIHEISKMVIKYNAIIAM |
| | | EDLSYGFKKGRFKVERQVYQKFETMLINKL |
| | | NYLVFKDISITENGGLLKGYQLTYIPDKLK |
| | | NVGHQCGCIFYVPAAYTSKIDPTTGFVNIF |
| | | KFKDLTVDAKREFIKKFDSIRYDSEKNLFC |
| | | FTFDYNNFITQNTVMSKSSWSVYTYGVRIK |
| | | RRFVNGRFSNESDTIDITKDMEKTLEMTDI |
| | | NWRDGHDLRQDIIDYEIVQHIFEIFRLTVQ |
| | | MRNSLSELEDRDYDRLISPVLNENNIFYDS |
| | | AKAGDALPKDADANGAYCIALKGLYEIKQI |
| | | TENWKEDGKFSRDKLKISNKDWFDFIQNKR |
| | | YL |

### Definitions

To aid in understanding the compounds and methods as disclosed herein, several terms are defined below.

The use of the terms “a” and “an” and “the” and similar referents in the context of describing the compounds and methods as disclosed herein (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “Alt-R,” as that term may modify a Cas agent, such as a Cas protein, a Cas guide RNA, including a crRNA, tracrRNA, or sgRNA, or a Cas RNP complex refers to a trademarked (™ or ®) IDT product that is a purified, synthetic molecule of defined sequence composition and length and often include chemical modifications to help increase activity, nuclease resistance, and reduce innate immune responses compared to other Cas agents. Alt-R Cas proteins and enzymes include nuclear localization sequences (NLSs) and a terminal affinity tag, such as a one or more C-terminal 6-His tags.

The term “NHEJ” specifically refer to classical nonhomologous end joining.

The terms “MMEJ” and “Alt-NHEJ” refers to microhomology mediated end-joining.

The phrase “MMEJ inhibition” refers to polymerase theta-mediated end joining (TMEJ).

The terms “target DNA,” “DNA target,” and “DNA target site” refers to the specific DNA site at which a targeting DNA endonuclease contacts and initiates cleavage for the purpose of recombining the DNA site with a donor DNA.

The term “targeting DNA endonuclease” refers to a DNA endonuclease that is directed to a specific DNA site for initiating cleavage of the specific DNA site. An example of a targeting DNA endonuclease includes a CRISPR-associated endonuclease.

The term “genome editing agents” refers to components required for introducing a donor DNA into a specific DNA site using a targeting endonuclease. Genome editing agents include a targeting endonuclease and a donor DNA.

The term “recombination enhancing agent” refer to one or more molecules that improve homology directed recombination by one of several possible mechanisms, including inhibiting 53BP1-mediated DNA end protection, inhibiting microhomology-mediated end joining (MMEJ), and/or inhibiting non-homologous end joining (NHEJ).

The term “Alt-R HDR Enhancer” refers to a small molecule that effectively diverts the double strand break repair pathway, significantly increasing HDR efficiency in many common cell lines. As described herein, two versions of such Alt-R HDR Enhancer molecules are described, including Alt-R HDR Enhancer V1 and Alt-R HDR Enhancer V2.

The term “CRISPR” refers to Clustered Regularly Interspaced Short Palindromic Repeat bacterial adaptive immune system.

The terms “Cas” and “Cas endonuclease” generally refers to a CRISPR-associated endonuclease.

The term “Cas protein” generally refers to a wild-type protein, including a variant thereof, of a CRISPR-associated endonuclease (including the interchangeable terms Cas and Cas endonuclease).

The term “Cas nucleic acid” generally refers to a nucleic acid of a CRISPR-associated endonuclease, including a guide RNA, sgRNA, crRNA, or tracrRNA.

The terms “AsCpf1” or “AsCas12a” refers generally to a Cas protein or Cas12a nuclease derived from Acidaminococcus sp.

The term “AsCas12a Ultra” refers to an engineered AsCas12a mutant that includes two point mutations, M537R and F870L. See Zhang, L., Zuris, J. A., Viswanathan, R., Edelstein, J. N., Turk, R., Thommandru, B., Rube, H. T., Glenn, S. E., Collingwood, M. A., Bode, N. M., Beaudoin, S. F., Lele, S., Scott, S. N., Wasko, K. M., Sexton, S., Borges, C. M., Schubert, M. S., Kurgan, G. L., McNeill, M. S., . . . Vakulskas, C. A. (2021). “AsCas12a ultra nuclease facilitates the rapid generation of therapeutic cell medicines.” Nature Communications, 12(1), 1-15. https://doi.org/10.1038/s41467-021-24017-8.

The terms “Cas9” and “CRISPR/Cas9” refer to the CRISPR-associated bacterial adaptive immune system of Streptococcus pyogenes. Examples of this system are disclosed in U.S. patent application Ser. Nos. 15/729,491 and 15/964,041, filed Oct. 10, 2017 and Apr. 26, 2018, respectively (Attorney Docket Nos. IDT01-009-US and IDT01-009-US-CIP, respectively), the contents of which are incorporated by reference herein.

The term “variant,” as that term modifies a protein (for example, ubiquitin), refers to a protein that includes at least one amino acid substitution of the reference, typically wild-type, protein amino acid sequence, additional amino acids (for example, such as an affinity tag or nuclear localization signal), or a combination thereof.

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid adverse effects, within the scope of sound judgment of the skilled artisan. For those skilled in the art, the effective amount, may be determined according to their knowledge and standard methodology of merely routine experimentation based on the present disclosure.

The term “enhanced HDR editing activity” refers to the presence of the at least one recombination enhancing agent increases HDR editing activity at the targeted genomic locus relative to HDR editing activity at the targeted genomic locus in the absence of the at least one recombination enhancing agent. In certain embodiments as disclosed herein the HDR editing activity in the presence of at least one recombination enhancing agent is enhanced by an amount selected from the group consisting of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% relative to HDR editing activity at the targeted genomic locus in the absence of the at least one recombination enhancing agent.

The term “polypeptide” refers to any linear or branched peptide comprising more than one amino acid. Polypeptide includes protein or fragment thereof or fusion thereof, provided such protein, fragment or fusion retains a useful biochemical or biological activity.

A fusion protein typically includes extra amino acid information that is not native to the protein to which the extra amino acid information is covalently attached. Such extra amino acid information may include tags that enable purification or identification of the fusion protein. Such extra amino acid information may include peptides that enable the fusion proteins to be transported into cells and/or transported to specific locations within cells. Examples of tags for these purposes include the following: AviTag, which is a peptide allowing biotinylation by the enzyme BirA so the protein can be isolated by streptavidin (GLNDIFEAQKIEWHE; SEQ ID NO: 52); Calmodulin-tag, which is a peptide bound by the protein calmodulin (KRRWKKNFIAVSAANRFKKISSSGAL; SEQ ID NO: 53); polyglutamate tag, which is a peptide binding efficiently to anion-exchange resin such as Mono-Q (EEEEEE; SEQ ID NO: 54); E-tag, which is a peptide recognized by an antibody (GAPVPYPDPLEPR; SEQ ID NO: 55); FLAG-tag, which is a peptide recognized by an antibody (DYKDDDDK; SEQ ID NO: 56); HA-tag, which is a peptide from hemagglutinin recognized by an antibody (YPYDVPDYA; SEQ ID NO: 57); His-tag, which is typically 5-10 histidines bound by a nickel or cobalt chelate (HHHHHH; SEQ ID NO: 58); Myc-tag, which is a peptide derived from c-myc recognized by an antibody (EQKLISEEDL; SEQ ID NO: 59); NE-tag, which is a novel 18-amino-acid synthetic peptide (TKENPRSNQEESYDDNES; SEQ ID NO: 60) recognized by a monoclonal IgG1 antibody, which is useful in a wide spectrum of applications including Western blotting, ELISA, flow cytometry, immunocytochemistry, immunoprecipitation, and affinity purification of recombinant proteins; S-tag, which is a peptide derived from Ribonuclease A (KETAAAKFERQHMDS; SEQ ID NO: 61); SBP-tag, which is a peptide which binds to streptavidin; (MDEKTTGWRGGHVVEGLAGELEQLRARLEHHPQGQREP; SEQ ID NO: 62); Softag 1, which is intended for mammalian expression (SLAELLNAGLGGS; SEQ ID NO: 63); Softag 3, which is intended for prokaryotic expression (TQDPSRVG; SEQ ID NO: 64); Strep-tag, which is a peptide which binds to streptavidin or the modified streptavidin called streptactin (Strep-tag II: WSHPQFEK; SEQ ID NO: 65); TC tag, which is a tetracysteine tag that is recognized by FlAsH and ReAsH biarsenical compounds (CCPGCC; SEQ ID NO: 66); V5 tag, which is a peptide recognized by an antibody (GKPIPNPLLGLDST; SEQ ID NO: 67); VSV-tag, a peptide recognized by an antibody (YTDIEMNRLGK; SEQ ID NO: 68); Xpress tag (DLYDDDDK; SEQ ID NO: 69); Isopeptag, which is a peptide which binds covalently to pilin-C protein (TDKDMTITFTNKKDAE; SEQ ID NO: 70); SpyTag, which is a peptide which binds covalently to SpyCatcher protein (AHIVMVDAYKPTK; SEQ ID NO: 71); SnoopTag, a peptide which binds covalently to SnoopCatcher protein (KLGDIEFIKVNK; SEQ ID NO: 72); BCCP (Biotin Carboxyl Carrier Protein), which is a protein domain biotinylated by BirA to enable recognition by streptavidin; Glutathione-S-transferase-tag, which is a protein that binds to immobilized glutathione; Green fluorescent protein-tag, which is a protein which is spontaneously fluorescent and can be bound by antibodies; HaloTag, which is a mutated bacterial haloalkane dehalogenase that covalently attaches to a reactive haloalkane substrate to allow attachment to a wide variety of substrates; Maltose binding protein-tag, a protein which binds to amylose agarose; Nus-tag; Thioredoxin-tag; and Fc-tag, derived from immunoglobulin Fc domain, which allows dimerization and solubilization and can be used for purification on Protein-A Sepharose. Nuclear localization signals (NLS), such as those obtained from SV40, allow for proteins to be transported to the nucleus immediately upon entering the cell. Given that the native Cas9 protein is bacterial in origin and therefore does not naturally comprise a NLS motif, addition of one or more NLS motifs to the recombinant Cas9 protein is expected to show improved genome editing activity when used in eukaryotic cells where the target genomic DNA substrate resides in the nucleus. One skilled in the art would appreciate these various fusion tag technologies, as well as how to make and use fusion proteins that include them.

The terms “tag-free” or “tagless” as that term modifies a polypeptide refers to a polypeptide lacking extra amino acid information that is not native to the polypeptide.

The terms “Ubiquitin” or “human Ubiquitin” refers to the wild-type Ubiquitin polypeptide amino acid sequence.

The term “Ubv-A molecule” refers to a Ubiquitin variant polypeptide with the amino acid sequence corresponding to the CM1/Ubv-A mutant polypeptide or a nucleic acid encoding the same.

The terms “i53,” i53 Ubiquitin,” or “Ubiquitin i53” refers to a ubiquitin variant polypeptide amino acid sequence that lacks the carboxy terminal di-glycine of the wild-type Ubiquitin polypeptide and includes several amino acid substitutions (Q2L, I44A, Q49S, Q62L, E64D, T66K, L69P, and V70L) relative to the wild-type Ubiquitin polypeptide.

### REFERENCES

1. Orthwein, A. et al. A mechanism for the suppression of homologous recombination in G1 cells. Nature 528, 422-426 (2015).
2. Chapman, J. R., Taylor, M. R. & Boulton, S. J. Playing the end game: DNA double-strand break repair pathway choice. Molecular cell 47, 497-510 (2012).
3. Ceccaldi, R. et al. Homologous-recombination-deficient tumours are dependent on Poltheta-mediated repair. Nature 518, 258-262 (2015).
4. Brambati, A. et al. RHINO restricts MMEJ activity to mitosis. bioRxiv (2023).
5. Xue, C. & Greene, E. C. DNA Repair Pathway Choices in CRISPR-Cas9-Mediated Genome Editing. Trends Genet 37, 639-656 (2021).
6. Seol, J. H., Shim, E. Y. & Lee, S. E. Microhomology-mediated end joining: Good, bad and ugly. Mutat Res 809, 81-87 (2018).
7. Shen, M. W. et al. Predictable and precise template-free CRISPR editing of pathogenic variants. Nature 563, 646-651 (2018).
8. Ranjha, L., Howard, S. M. & Cejka, P. Main steps in DNA double-strand break repair: an introduction to homologous recombination and related processes. Chromosoma 127, 187-214 (2018).
9. Nimonkar, A. V. et al. BLM-DNA2-RPA-MRN and EXO1-BLM-RPA-MRN constitute two DNA end resection machineries for human DNA break repair. Genes & development 25, 350-362 (2011).
10. Chaplin, A. K. et al. Dimers of DNA-PK create a stage for DNA double-strand break repair. Nat Struct Mol Biol 28, 13-19 (2021).
11. Chen, S., Lees-Miller, J. P., He, Y. & Lees-Miller, S. P. Structural insights into the role of DNA-PK as a master regulator in NHEJ. Genome Instab Dis 2, 195-210 (2021).
12. Ghosh, D. & Raghavan, S. C. Nonhomologous end joining: new accessory factors fine tune the machinery. Trends Genet 37, 582-599 (2021).
13. Robert, F., Barbeau, M., Ethier, S., Dostie, J. & Pelletier, J. Pharmacological inhibition of DNA-PK stimulates Cas9-mediated genome editing. Genome Med 7, 93 (2015).
14. Riesenberg, S. & Maricic, T. Targeting repair pathways with small molecules increases precise genome editing in pluripotent stem cells. Nat Commun 9, 2164 (2018).
15. Riesenberg, S. et al. Simultaneous precise editing of multiple genes in human cells. Nucleic Acids Res 47, e116 (2019).
16. Wimberger, S. et al. Simultaneous inhibition of DNA-PK and PolΘ improves integration efficiency and precision of genome editing. Nat Commun 14, 4761 (2023).
17. Maruyama, T. et al. Increasing the efficiency of precise genome editing with CRISPR-Cas9 by inhibition of nonhomologous end joining. Nature biotechnology 33, 538-542 (2015).
18. DeWitt, M. A. et al. Selection-free genome editing of the sickle mutation in human adult hematopoietic stem/progenitor cells. Sci Transl Med 8, 360ra134 (2016).
19. Gavande, N. S. et al. Discovery and development of novel DNA-PK inhibitors by targeting the unique Ku-DNA interaction. Nucleic Acids Res 48, 11536-11550 (2020).
20. Hussmann, J. A. et al. Mapping the genetic landscape of DNA double-strand break repair. Cell 184, 5653-5669 e5625 (2021).
21. Truong, L. N. et al. Microhomology-mediated End Joining and Homologous Recombination share the initial end resection step to repair DNA double-strand breaks in mammalian cells. Proceedings of the National Academy of Sciences of the United States of America 110, 7720-7725 (2013).
22. Kent, T., Chandramouly, G., McDevitt, S. M., Ozdemir, A. Y. & Pomerantz, R. T. Mechanism of microhomology-mediated end-joining promoted by human DNA polymerase theta. Nat Struct Mol Biol 22, 230-237 (2015).
23. Patterson-Fortin, J. & D'Andrea, A. D. Exploiting the Microhomology-Mediated End-Joining Pathway in Cancer Therapy. Cancer Res 80, 4593-4600 (2020).
24. Liang, L. et al. Human DNA ligases I and III, but not ligase IV, are required for microhomology-mediated end joining of DNA double-strand breaks. Nucleic Acids Res 36, 3297-3310 (2008).
25. Arai, D. & Nakao, Y. Efficient biallelic knock-in in mouse embryonic stem cells by in vivo-linearization of donor and transient inhibition of DNA polymerase theta/DNA-PK. Sci Rep 11, 18132 (2021).
26. Riesenberg, S. et al. Efficient high-precision homology-directed repair-dependent genome editing by HDRobust. Nat Methods (2023).
27. Schimmel, J. et al. Modulating mutational outcomes and improving precise gene editing at CRISPR-Cas9-induced breaks by chemical inhibition of end-joining pathways. Cell reports 42, 112019 (2023).
28. Stockley, M. L. et al. Discovery, Characterization, and Structure-Based Optimization of Small-Molecule In Vitro and In Vivo Probes for Human DNA Polymerase Theta. J Med Chem 65, 13879-13891 (2022).
29. Fok, J. H. L. et al. AZD7648 is a potent and selective DNA-PK inhibitor that enhances radiation, chemotherapy and olaparib activity. Nat Commun 10, 5065 (2019).
30. Bubenik, M. et al. Identification of RP-6685, an Orally Bioavailable Compound that Inhibits the DNA Polymerase Activity of Poltheta. J Med Chem 65, 13198-13215 (2022).
31. Zatreanu, D. et al. Poltheta inhibitors elicit BRCA-gene synthetic lethality and target PARP inhibitor resistance. Nat Commun 12, 3636 (2021).
32. Zhou, J. et al. A first-in-class Polymerase Theta Inhibitor selectively targets Homologous-Recombination-Deficient Tumors. Nat Cancer 2, 598-610 (2021).
33. Iwabuchi, K., Bartel, P. L., Li, B., Marraccino, R. & Fields, S. Two cellular proteins that bind to wild-type but not mutant p53. Proceedings of the National Academy of Sciences of the United States of America 91, 6098-6102 (1994).
34. Escribano-Díaz, C. et al. A cell cycle-dependent regulatory circuit composed of 53BP1-RIF1 and BRCA1-CtIP controls DNA repair pathway choice. Molecular cell 49, 872-883 (2013).
35. Panier, S. & Boulton, S. J. Double-strand break repair: 53BP1 comes into focus. Nature reviews. Molecular cell biology 15, 7-18 (2014).
36. Botuyan, M. V. et al. Structural basis for the methylation state-specific recognition of histone H4-K20 by 53BP1 and Crb2 in DNA repair. Cell 127, 1361-1373 (2006).
37. Charier, G. et al. The Tudor tandem of 53BP1: a new structural motif involved in DNA and RG-rich peptide binding. Structure (London, England: 1993) 12, 1551-1562 (2004).
38. Fradet-Turcotte, A. et al. 53BP1 is a reader of the DNA-damage-induced H2A Lys 15 ubiquitin mark. Nature 499, 50-54 (2013).
39. Al-Hakim, A. et al. The ubiquitous role of ubiquitin in the DNA damage response. DNA Repair (Amst) 9, 1229-1240 (2010).
40. Doil, C. et al. RNF168 binds and amplifies ubiquitin conjugates on damaged chromosomes to allow accumulation of repair proteins. Cell 136, 435-446 (2009).
41. Mallette, F. A. et al. RNF8- and RNF168-dependent degradation of KDM4A/JMJD2A triggers 53BP1 recruitment to DNA damage sites. Embo j 31, 1865-1878 (2012).
42. Acs, K. et al. The AAA-ATPase VCP/p97 promotes 53BP1 recruitment by removing L3MBTL1 from DNA double-strand breaks. Nat Struct Mol Biol 18, 1345-1350 (2011).
43. Mattiroli, F. et al. RNF168 ubiquitinates K13-15 on H2A/H2AX to drive DNA damage signaling. Cell 150, 1182-1195 (2012).
44. Chapman, J. R. et al. RIF1 is essential for 53BP1-dependent nonhomologous end joining and suppression of DNA double-strand break resection. Molecular cell 49, 858-871 (2013).
45. Callen, E. et al. 53BP1 mediates productive and mutagenic DNA repair through distinct phosphoprotein interactions. Cell 153, 1266-1280 (2013).
46. Noordermeer, S. M. et al. The shieldin complex mediates 53BP1-dependent DNA repair. Nature 560, 117-121 (2018).
47. Mirman, Z. et al. 53BP1-RIF1-shieldin counteracts DSB resection through CST- and Polalpha-dependent fill-in. Nature 560, 112-116 (2018).
48. Wang, J. et al. PTIP associates with Artemis to dictate DNA repair pathway choice. Genes & development 28, 2693-2698 (2014).
49. Canny, M. D. et al. Inhibition of 53BP1 favors homology-dependent DNA repair and increases CRISPR-Cas9 genome-editing efficiency. Nature biotechnology 36, 95-102 (2018).
50. Bunting, S. F. et al. 53BP1 inhibits homologous recombination in Brca1-deficient cells by blocking resection of DNA breaks. Cell 141, 243-254 (2010).
51. Boersma, V. et al. MAD2L2 controls DNA repair at telomeres and DNA breaks by inhibiting 5′ end resection. Nature 521, 537-540 (2015).
52. Perfetti, M. T. et al. Identification of a fragment-like small molecule ligand for the methyl-lysine binding protein, 53BP1. ACS Chem Biol 10, 1072-1081 (2015).
53. Sun, Y. et al. Discovery of a novel 53BP1 inhibitor through AlphaScreen-based high-throughput screening. Bioorg Med Chem 34, 116054 (2021).
54. Xie, A. et al. Distinct roles of chromatin-associated proteins MDC1 and 53BP1 in mammalian double-strand break repair. Molecular cell 28, 1045-1057 (2007).
55. Paulsen, B. S. et al. Ectopic expression of RAD52 and dn53BP1 improves homology-directed repair during CRISPR-Cas9 genome editing. Nat Biomed Eng 1, 878-888 (2017).
56. Nambiar, T. S. et al. Stimulation of CRISPR-mediated homology-directed repair by an engineered RAD18 variant. Nat Commun 10, 3395 (2019).
57. Gupta, R. et al. DNA Repair Network Analysis Reveals Shieldin as a Key Regulator of NHEJ and PARP Inhibitor Sensitivity. Cell 173, 972-988 e923 (2018).
58. Jimeno, S. et al. Neddylation inhibits CtIP-mediated resection and regulates DNA double strand break repair pathway choice. Nucleic Acids Res 43, 987-999 (2015).
59. Bertino, E. M. & Otterson, G. A. Romidepsin: a novel histone deacetylase inhibitor for cancer. Expert Opinion on Investigational Drugs 20, 1151-1158 (2011).
60. Zhang, J. P. et al. HDAC inhibitors improve CRISPR-mediated HDR editing efficiency in iPSCs. Sci China Life Sci 64, 1449-1462 (2021).
61. Li, G. et al. Increasing CRISPR/Cas9-mediated homology-directed DNA repair by histone deacetylase inhibitors. Int J Biochem Cell Biol 125, 105790 (2020).
62. Tang, J. et al. Acetylation limits 53BP1 association with damaged chromatin to promote homologous recombination. Nat Struct Mol Biol 20, 317-325 (2013).
63. Hsiao, K. Y. & Mizzen, C. A. Histone H4 deacetylation facilitates 53BP1 DNA damage signaling and double-strand break repair. J Mol Cell Biol 5, 157-165 (2013).
64. Ma, T. et al. RNF111-dependent neddylation activates DNA damage-induced ubiquitination. Molecular cell 49, 897-907 (2013).
65. Chen, J. et al. 53BP1 loss rescues embryonic lethality but not genomic instability of BRCA1 total knockout mice. Cell Death Differ 27, 2552-2567 (2020).
66. Schubert, M. S. et al. Optimized design parameters for CRISPR Cas9 and Cas12a homology-directed repair. Sci Rep 11, 19482 (2021).
67. Dobosy, J. R. et al. RNase H-dependent PCR (rhPCR): improved specificity and single nucleotide polymorphism detection using blocked cleavable primers. BMC Biotechnology 11, 80 (2011).
68. Kurgan, G. et al. CRISPAltRations: a validated cloud-based approach for interrogation of double-strand break repair mediated by CRISPR genome editing. Mol Ther Methods Clin Dev 21, 478-491 (2021).

All references, including publications, patent applications, and patents, cited herein are hereby incorporated by reference to the same extent as if each reference were individually and specifically indicated to be incorporated by reference and were set forth in its entirety herein.

Preferred embodiments of the compounds and methods as disclosed herein are as described herein, including the best mode known to the inventors for carrying out the compounds and methods as disclosed herein. Variations of those preferred embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A composition for enhancing homology-directed repair (HDR) editing activity in a cell comprising:
a. a first recombination enhancing agent that inhibits 53BP1-mediated DNA end protection; and
b. at least one additional recombination enhancing agent selected from the group consisting of a second recombination enhancing agent which inhibits microhomology-mediated end joining (MMEJ) and a third recombination enhancing agent which inhibits non-homologous end joining (NHEJ), or combinations thereof.

2. The composition of claim 1, wherein the first recombination enhancing agent which inhibits 53BP1-mediated DNA end protection is a 53BP1 binding ubiquitin variant.

3. The composition of claim 1, wherein the first recombination enhancing agent which inhibits 53BP1-mediated DNA end protection is a 53BP1 binding ubiquitin variant selected from the group consisting of a Ubv-A polypeptide; an mRNA encoding a Ubv-A polypeptide; and a nucleic acid encoding a Ubv-A polypeptide.

4. The composition of claim 1, wherein the first recombination enhancing agent which inhibits 53BP1-mediated DNA end protection is selected from the group consisting of a Ubv-A polypeptide comprising a sequence having 90%, 95%, or 100% identity to SEQ ID NO: 1; an mRNA encoding a Ubv-A polypeptide comprising a sequence having 90%, 95%, or 100% identity to SEQ ID NO: 3; and a nucleic acid encoding a Ubv-A polypeptide comprising a sequence having 90%, 95%, or 100% identity to SEQ ID NO: 2.

5. The composition of claim 1, wherein the second recombination enhancing agent which inhibits microhomology-mediated end joining (MMEJ) is selected from the group consisting of an ART558 molecule, RP-6685, Novobiocin, PolQi1, and PolQi2.

6. The composition of claim 1, wherein the third recombination enhancing agent which inhibits non-homologous end joining (NHEJ) is selected from the group consisting of NU7441, NU7026, KU-0060648, M3841, CC-115, SCR7, AZD7648 and an Alt-R HDR enhancer V2 molecule.

7. The composition of claim 1, further comprising a compound selected from 53BP1 inhibitors, HDAC inhibitors, and combinations thereof.

8. The composition of claim 7, wherein the 53BP1 inhibitor is selected from the group consisting of inhibitors of proteins involved in 53BP1 mediated end-protection including but not limited to 53BP1, MDC1, RNF8, RNF168, RIF1, PTIP, Artemis, REV7, SHLD1, SHLD2, SHLD3, CTC1, STN1, TEN1, and combinations thereof.

9. The composition of claim 1, further comprising a CRISPR-associated protein selected from the group consisting of Cas9 wild-type, a Cas9 variant, SpCas9, Cas12a wild-type, a Cas12 variant, AsCas12a, AsCas12a Ultra, ErCas12a, EURECA-V, and combinations thereof.

10. The composition of claim 1, further comprising a nucleic acid encoding a CRISPR-associated protein selected from the group consisting of Cas9 wild-type, a Cas9 variant, SpCas9, Cas12a wild-type, a Cas12 variant, AsCas12a, AsCas12a Ultra, ErCas12a, EURECA-V, and combinations thereof.

11. The composition of claim 1, further comprising a guide RNA selected from a single guide RNA (sgRNA) or a combination of a CRISPR RNA (crRNA) and a trans-activating CRISPR RNA (tracrRNA).

12. The composition of claim 1, further comprising a donor DNA template.

13. A method for enhancing homology-directed repair (HDR) editing activity in a cell, the method comprising:
a. introducing into the cell a first recombination enhancing agent that inhibits 53BP1-mediated DNA end protection; and
b. introducing into the cell at least one additional recombination enhancing agent selected from the group consisting of a second recombination enhancing agent which inhibits microhomology-mediated end joining (MMEJ) and a third recombination enhancing agent which inhibits non-homologous end joining (NHEJ), or combinations thereof,
wherein the presence of the at least one recombination enhancing agent increases HDR editing activity at the targeted genomic locus relative to HDR editing activity at the targeted genomic locus in the absence of the at least one recombination enhancing agent.

14. The method of claim 13, wherein the first recombination enhancing agent which inhibits 53BP1-mediated DNA end protection is a 53BP1 binding ubiquitin variant.

15. The method of claim 13, wherein the first recombination enhancing agent which inhibits 53BP1-mediated DNA end protection is a 53BP1 binding ubiquitin variant selected from the group consisting of a Ubv-A polypeptide; an mRNA encoding a Ubv-A polypeptide; and a nucleic acid encoding a Ubv-A polypeptide.

16. The method of claim 13, wherein the first recombination enhancing agent which inhibits 53BP1-mediated DNA end protection is selected from the group consisting of a Ubv-A polypeptide comprising a sequence having 90%, 95%, or 100% identity to SEQ ID NO: 1; an mRNA encoding a Ubv-A polypeptide comprising a sequence having 90%, 95%, or 100% identity to SEQ ID NO: 3; and a nucleic acid encoding a Ubv-A polypeptide comprising a sequence having 90%, 95%, or 100% identity to SEQ ID NO: 2.

17. The method of claim 13, further comprising introducing into the cell a CRISPR-associated protein selected from the group consisting of Cas9 wild-type, a Cas9 variant, SpCas9, Cas12a wild-type, a Cas12 variant, AsCas12a, AsCas12a Ultra, ErCas12a, EURECA-V, and combinations thereof.

18. The method of claim 13, further comprising introducing into the cell a nucleic acid encoding a CRISPR-associated protein selected from the group consisting of Cas9 wild-type, a Cas9 variant, SpCas9, Cas12a wild-type, a Cas12 variant, AsCas12a, AsCas12a Ultra, ErCas12a, EURECA-V, and combinations thereof.

19. The method of claim 13, further comprising introducing into the cell a guide RNA selected from a single guide RNA (sgRNA) or a combination of a CRISPR RNA (crRNA) and a trans-activating CRISPR RNA (tracrRNA).

20. The method of claim 13, further comprising introducing into the cell a donor DNA template.


SEQUENCE LISTING (73 sequences)

SEQ ID NO: 1 - protein, 74 aa, synthetic construct
MLIFVRMLTGKMIELEVEPSDTIENVKAKIQDHEGIPPDQQRLAFQGKSLEDGRTLSDYNILKDPKKMPLLRLR

SEQ ID NO: 2 - DNA, 222 bp, synthetic construct
  source 1..222: mol_type = other DNA
atgttgattttcgtacgcatgttgactggaaagatgatcgagttggaagtggagccttccgatactatcgagaatgttaaggccaaaatccaagatcatgaagggattcctccagatcaacaacgccttgcttttcaagggaagagcctggaggacggtcgcacactgtctgactataacattcttaaagatcctaaaaagatgccactgctgcgcttgcgt

SEQ ID NO: 3 - RNA, 425 nt, synthetic construct
  source 1..425: mol_type = other RNA
gggagacccaagctggctagcgtttaaacgggccctctagactcgagcggccgccaccatgctgatcttcgtgagaatgctgaccggcaagatgatcgaactggaagtggaacccagcgacaccatcgagaacgtgaaggccaaaatccaggaccacgagggcatccctcctgaccagcagagactggcctttcagggaaagtccctggaagatggaagaaccctgagcgactacaacatcctgaaggaccctaagaagatgccactgctgagactgagatgatcagcctcgactgtgccttctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 21 bp, synthetic construct
  source 1..21: mol_type = other DNA
gaggtcacaaaacccagtagg

SEQ ID NO: 5 - DNA, 21 bp, synthetic construct
  source 1..21: mol_type = other DNA
aacagactgatggttcccatt

SEQ ID NO: 6 - DNA, 21 bp, synthetic construct
  source 1..21: mol_type = other DNA
ctggtggattttgaatttggg

SEQ ID NO: 7 - DNA, 21 bp, synthetic construct
  source 1..21: mol_type = other DNA
cgtggttgctcccgcaagttt

SEQ ID NO: 8 - DNA, 21 bp, synthetic construct
  source 1..21: mol_type = other DNA
tttacttcttgacggtccaaa

SEQ ID NO: 9 - DNA, 20 bp, synthetic construct
  source 1..20: mol_type = other DNA
ggcatcgtgtactaccacgg

SEQ ID NO: 10 - DNA, 20 bp, synthetic construct
  source 1..20: mol_type = other DNA
tcggaaggcatatatcgtca

SEQ ID NO: 11 - DNA, 20 bp, synthetic construct
  source 1..20: mol_type = other DNA
ctggtaagtgtagtagacga

SEQ ID NO: 12 - DNA, 20 bp, synthetic construct
  source 1..20: mol_type = other DNA
ctgggacgcaacctctctcg

SEQ ID NO: 13 - DNA, 20 bp, synthetic construct
  source 1..20: mol_type = other DNA
acgccaatggcaactacact

SEQ ID NO: 14 - DNA, 20 bp, synthetic construct
  source 1..20: mol_type = other DNA
gagaatcaaaatcggtgaat

SEQ ID NO: 15 - DNA, 91 bp, synthetic construct
  source 1..91: mol_type = other DNA
gaggtcattcctgtgagtcctttggaggtcacaaaacccagaattcgtaggggcaggcaaggggaaagctcacccctcttacctttttcca

SEQ ID NO: 16 - DNA, 91 bp, synthetic construct
  source 1..91: mol_type = other DNA
ctccttccagcacctcataatttgaacagactgatggttcgaattcccattagtcacataaagctgtagtctagtacagacgtccttagaa

SEQ ID NO: 17 - DNA, 91 bp, synthetic construct
  source 1..91: mol_type = other DNA
gctgactgcctgccctgtgttttcctggtggattttgaatgaattcttgggtgtgctacatcatacattcttccagaacttcaggatgggc

SEQ ID NO: 18 - DNA, 91 bp, synthetic construct
  source 1..91: mol_type = other DNA
ttgaggctgtcagagcgctttttgcgtggttgctcccgcagaattcagtttccttctctggagcttcccgcaggtgggcagctagctgcag

SEQ ID NO: 19 - DNA, 91 bp, synthetic construct
  source 1..91: mol_type = other DNA
atgcctttgaaagcaacaattttatttacttcttgacggtgaattcccaaagggaaactctagatgctcagacttttcacacaagaataat

SEQ ID NO: 20 - DNA, 86 bp, synthetic construct
  source 1..86: mol_type = other DNA
catccacctgcaccctgaagcctggcatcgtgtactaccagaattccggtggcgggggcgtcatggggagtttgagtaagaaccat

SEQ ID NO: 21 - DNA, 86 bp, synthetic construct
  source 1..86: mol_type = other DNA
cgaaaaataagtttagatgaccttcggaaggcatatatcggaattctcaaggatgttcagcagtacattcttcatcgtttagatca

SEQ ID NO: 22 - DNA, 86 bp, synthetic construct
  source 1..86: mol_type = other DNA
gggaggcttcacctttggaagcactggtaagtgtagtagagaattccgagggtgaacggggagatgatgagcttgctggccatgct

SEQ ID NO: 23 - DNA, 86 bp, synthetic construct
  source 1..86: mol_type = other DNA
ggcggccacggcggctccaggctctgggacgcaacctctcgaattctcggggtggcactccagggccgactgcggctgtgaaggtt

SEQ ID NO: 24 - DNA, 86 bp, synthetic construct
  source 1..86: mol_type = other DNA
gcgcattctgcaaggtctgcaggacgccaatggcaactacgaattcactaggcagacggacattctgcaagctttctctgaagaga

SEQ ID NO: 25 - DNA, 3073 bp, synthetic construct
  source 1..3073: mol_type = other DNA
gtcgtaccgactggtagatgacagcaaaccccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgccaccaacttctctcttttgaagcaggccggagatgtggaagaaaatcctgggcccatggctcttcctgtgactgcccttctgctgcccctggctctcctgctccatgctgcccggccagacatccagatgacacagacaaccagcagcctcagcgccagcctgggggacagagtgaccattagctgccgggcctctcaggacatcagcaaatacctgaactggtaccagcagaaaccagatggcactgtcaagctgctgatttaccacacctccaggctccacagcggcgtgcccagtcgcttcagcggcagtgggagcgggacagattattccctcacaatctccaacctggagcaggaagatattgccacatacttctgccagcaaggcaacaccctgccatacacatttggaggcggcaccaaattggagatcaccggcggtggtggatctggaggagggggcagcggaggtggcggctctgaggtgaaactgcaggagagtggccctggcctggtggctcccagccagagcctttctgtcacctgcaccgtgtctggggtgtccctgcctgactatggagtctcctggatccggcagcctccaagaaaaggactggaatggctgggcgtcatctggggaagtgagaccacctactataattcagccctcaagtcccggctcaccatcattaaggacaactccaaatcccaggtgttcctgaagatgaattctctccagactgatgacacagccatctactactgtgccaagcattattattacggcgggtcctatgccatggactactggggccaggggaccagtgtcactgtttcttctgaaagcaagtatggccccccatgccctccctgccccggacagccgcgggagccccaggtctacaccctcccaccctccagagatgagctgaccaagaaccaggtttcactgacatgcctggtgaagggcttctacccctctgacattgctgtggagtgggagagcaatgggcagccagagaacaactacaagaccacacctcctgtcctggacagcgacggctccttcttcctctacagtaagctcactgtggacaagagccgctggcagcagggcaatgtcttctcctgcagcgtgatgcacgaggccctgcacaatgcctatacccagaagtcactctctctgagccctggaaagaaggatcccaagttttgggtcctcgtggtggtcgggggtgtgctggcctgctacagcttgctggtcacagtggccttcatcatcttctgggtgcgctccaagaggagccggctgcttcacagtgattacatgaacatgacccccaggaggccaggacccaccaggaagcactaccagccctacgctcccccgcgggactttgctgcttaccgcagcagggtcaaattttctagatctgcagatgcgccggcctatcaacaaggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcgttaaattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcgatccgaaaacgggcgtatagtcgagacc

SEQ ID NO: 26 - DNA, 28 bp, synthetic construct
  source 1..28: mol_type = other DNA
  misc_difference 24: note = ribose moiety
aggtctttgactgtaactaccagggaga

SEQ ID NO: 27 - DNA, 27 bp, synthetic construct
  source 1..27: mol_type = other DNA
  misc_difference 23: note = ribose moiety
gaagattagcggccatgtattccaatg

SEQ ID NO: 28 - DNA, 28 bp, synthetic construct
  source 1..28: mol_type = other DNA
  misc_difference 24: note = ribose moiety
ctcactcatttttcaaaacacgcataaa

SEQ ID NO: 29 - DNA, 27 bp, synthetic construct
  source 1..27: mol_type = other DNA
  misc_difference 23: note = ribose moiety
actatttcagccaacaagaagtgtcac

SEQ ID NO: 30 - DNA, 29 bp, synthetic construct
  source 1..29: mol_type = other DNA
  misc_difference 25: note = ribose moiety
tagatggtgtcatttttggcaatagagac

SEQ ID NO: 31 - DNA, 28 bp, synthetic construct
  source 1..28: mol_type = other DNA
  misc_difference 24: note = ribose moiety
gaaaagtatgggtgttctctttggagga

SEQ ID NO: 32 - DNA, 24 bp, synthetic construct
  source 1..24: mol_type = other DNA
  misc_difference 21: note = ribose moiety
agacagactgtgagcctagcaggg

SEQ ID NO: 33 - DNA, 25 bp, synthetic construct
  source 1..25: mol_type = other DNA
  misc_difference 21: note = ribose moiety
agagttcaacaggctgtgatgatgc

SEQ ID NO: 34 - DNA, 30 bp, synthetic construct
  source 1..30: mol_type = other DNA
  misc_difference 26: note = ribose moiety
gaccagtcctacattgatgttttacctgag

SEQ ID NO: 35 - DNA, 29 bp, synthetic construct
  source 1..29: mol_type = other DNA
  misc_difference 25: note = ribose moiety
gtaggaatgcaatccagagtttatggaac

SEQ ID NO: 36 - DNA, 25 bp, synthetic construct
  source 1..25: mol_type = other DNA
  misc_difference 21: note = ribose moiety
atttccgctttgggacaatccctgt

SEQ ID NO: 37 - DNA, 25 bp, synthetic construct
  source 1..25: mol_type = other DNA
  misc_difference 21: note = ribose moiety
gaagatgcccattcttccaagttat

SEQ ID NO: 38 - DNA, 29 bp, synthetic construct
  source 1..29: mol_type = other DNA
  misc_difference 25: note = ribose moiety
catctctgcagtttcttttgtgaggtgtt

SEQ ID NO: 39 - DNA, 31 bp, synthetic construct
  source 1..31: mol_type = other DNA
  misc_difference 27: note = ribose moiety
gttgacattctctttgaagatatggtacttc

SEQ ID NO: 40 - DNA, 25 bp, synthetic construct
  source 1..25: mol_type = other DNA
  misc_difference 21: note = ribose moiety
ttctcttggaccttgacctgggaga

SEQ ID NO: 41 - DNA, 23 bp, synthetic construct
  source 1..23: mol_type = other DNA
  misc_difference 19: note = ribose moiety
tgtgtcattggctccagggacca

SEQ ID NO: 42 - DNA, 22 bp, synthetic construct
  source 1..22: mol_type = other DNA
  misc_difference 18: note = ribose moiety
gttctccccaagcccatcgtag

SEQ ID NO: 43 - DNA, 23 bp, synthetic construct
  source 1..23: mol_type = other DNA
  misc_difference 19: note = ribose moiety
agctgagtcatcctcgtccggag

SEQ ID NO: 44 - DNA, 28 bp, synthetic construct
  source 1..28: mol_type = other DNA
  misc_difference 24: note = ribose moiety
tcttactctgtcaacgtgatagtggaca

SEQ ID NO: 45 - DNA, 29 bp, synthetic construct
  source 1..29: mol_type = other DNA
  misc_difference 25: note = ribose moiety
cattgcttaacttcaaggaaaacactcac

SEQ ID NO: 46 - DNA, 45 bp, synthetic construct
  source 1..45: mol_type = other DNA
tttctgttggtgctgatattgcgagagagcaatctcctggtaatg

SEQ ID NO: 47 - DNA, 44 bp, synthetic construct
  source 1..44: mol_type = other DNA
acttgcctgtcgctctatcttcctgacttagtgagctgggaaag

SEQ ID NO: 48 - protein, 1368 aa, synthetic construct [sequence omitted]

SEQ ID NO: 49 - DNA, 4104 bp, synthetic construct
  source 1..4104: mol_type = other DNA
atggacaaaaagtactctattggcctggatatcgggaccaacagcgtcgggtgggctgttatcaccgacgagtataaagtaccttcgaaaaagttcaaagtgctgggcaacaccgatcgccattcaatcaaaaagaacttgattggtgcgctgttgtttgactccggggaaaccgccgaggcgactcgccttaaacgtacagcacgtcgccggtacactcggcgtaagaatcgcatttgctatttgcaggaaatctttagcaacgagatggcaaaagtcgatgactcgtttttccaccgcctcgaggaaagctttctggtggaggaagacaaaaagcatgagcgtcacccgatcttcggcaacattgtcgatgaagtagcgtatcatgaaaaatacccaaccatttaccacttacgcaaaaagctggtggacagcactgacaaagctgatttgcgccttatctatttagccctggcacatatgattaagtttcgtggtcacttcctgatcgaaggagacttaaatcccgacaacagtgatgttgataaattgtttattcagcttgtccaaacttacaatcaactgttcgaggaaaacccgatcaatgcctccggtgtggatgcaaaagccattttaagtgcacgccttagcaagtcccgtcgcttagaaaaccttatcgcgcagctgcccggcgagaaaaagaatggtttgtttgggaaccttattgccttgagcttaggcctcaccccgaatttcaaaagtaatttcgatcttgcagaagacgccaaattacaactgtcgaaggatacttatgatgacgatctcgataatctgttagcgcagattggtgaccaatacgccgatctttttctggcggctaaaaatctgagcgacgccatcttgctttcggatattctccgcgttaacaccgaaatcacgaaagcgcctcttagtgccagcatgattaaacgttatgatgaacaccaccaggacctgaccttactcaaagcgttggttcgccagcaactgccagagaagtacaaagaaatcttctttgatcagtcaaagaatggttatgccggctatattgacgggggtgcaagccaagaggaattctacaaatttatcaagcctattctggagaaaatggatggcaccgaagagttattggtgaagcttaaccgtgaagacctcctgcggaaacagcgcacattcgataatggttcgatcccacaccaaatccatttgggggagttacacgctattttgcgtcgccaggaagacttttaccctttcctgaaggataaccgggagaaaattgagaagatccttacctttcgtattccgtattacgtaggccccttagcacggggtaatagccgtttcgcgtggatgacacggaagtcggaagagacgatcaccccgtggaacttcgaagaggtagtcgacaagggcgcatcagcgcagtcttttattgaacgtatgacgaatttcgataaaaacttgcccaatgagaaggtgcttccgaaacattccttgttatatgaatattttacagtttacaacgagctgaccaaggttaaatacgtgacggaaggaatgcgcaagcccgcttttcttagcggtgagcaaaaaaaggcgatcgtcgacctgttattcaaaacgaatcgtaaggtgactgtaaagcaactcaaagaagattacttcaaaaagattgagtgcttcgacagcgtcgaaatctctggggtagaggatcggtttaacgcaagtttaggtacctaccatgacctgcttaaaatcattaaggataaagacttcttagataatgaagagaacgaagatattctcgaggacatcgtcttgacgttaaccttatttgaggatcgtgaaatgattgaggaacgcctcaaaacttatgcccacctgttcgacgataaggtgatgaagcagctgaaacgtcggcgctacacaggatggggccgcttgagtcgcaaacttattaacggaatccgtgacaagcaatccggcaaaacgattctggatttcttgaagtcggacggatttgctaatcgcaacttcatgcagttgatccatgatgactccctgacttttaaagaggatattcaaaaggcgcaggttagtggtcaaggcgacagcttacacgaacacatcgcaaatttggctggttcgccggccattaaaaaggggatcctccagaccgtgaaagttgtagatgagcttgttaaggtcatgggtcgtcataagcccgaaaacatcgtgattgaaatggcgcgggagaatcaaacgacccagaaaggacaaaagaatagccgtgaacggatgaagcggatcgaggaaggcattaaagagctggggtctcaaatcttgaaggaacaccctgtggagaacactcagctccaaaatgaaaaactttacctgtactatttgcagaacggacgcgatatgtacgtggaccaagagttggatattaatcggctgagtgactacgacgttgatcatatcgtcccgcagagcttcctcaaagacgattctattgacaataaggtactgacgcgctctgataaaaaccgtggtaagtcggacaacgtgccctccgaagaggttgtgaaaaagatgaaaaattattggcgccagcttttaaacgcgaagctgatcacacaacgtaaattcgataatttgaccaaggctgaacggggtggcctgagcgagttagataaggcaggatttattaaacgccagttagtggagactcgtcaaatcaccaaacatgtcgcgcagattttggacagccggatgaacaccaagtacgatgaaaatgacaaactgatccgtgaggtgaaagtcattactctgaagtccaaattagttagtgatttccggaaggactttcaattctacaaagtccgtgaaattaataactatcatcacgcacatgacgcgtacctgaatgcagtggttgggaccgcccttatcaagaaatatcctaagctggagtcggagtttgtctatggcgactataaggtatacgatgttcgcaaaatgattgcgaaatctgagcaggagatcggtaaggcaaccgcaaaatatttcttttactcaaacattatgaatttctttaagacagaaatcactctggccaacggggagattcgcaaacgtccgttgatcgaaacaaacggcgagactggcgaaattgtttgggacaaagggcgtgatttcgcgacggtgcgcaaggtactgagcatgcctcaagtcaatattgttaagaaaaccgaagtgcagacgggcgggttttccaaggaaagcatcttacccaaacgtaattcagataaacttattgcacgcaaaaaggactgggatccgaaaaagtatggaggcttcgacagtccaaccgtagcctactctgttctcgttgtagcgaaagtagaaaagggtaaatccaagaaactgaaatctgtcaaggagttgcttggaatcaccattatggagcgtagctccttcgagaagaacccgattgactttctggaagccaaaggatataaagaggtcaagaaagatcttatcattaagctgcctaagtattcactcttcgagctggaaaatggtcgtaaacgcatgctcgcttctgccggcgagttgcagaagggcaatgaattagcacttccatcaaagtacgttaacttcctgtatttggccagccattacgagaaactgaaggggtctccagaggacaacgaacagaaacaattatttgtagagcagcacaagcattatcttgatgaaatcattgagcaaatttccgaattcagtaaacgcgtaatcctggccgatgcaaacctcgacaaggtgctgagcgcttacaataagcatcgcgacaaacctatccgtgagcaggctgaaaatatcattcacctgttcacattaacgaacctgggcgctccggccgcttttaaatatttcgacacgacaatcgaccgtaagcgctataccagtacgaaagaagtgttggatgcgacccttattcaccagtcaattacaggattatatgagacccgtatcgaccttagccaattaggtggggat

SEQ ID NO: 50 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 51 - DNA, 3921 bp, synthetic construct
  source 1..3921: mol_type = other DNA
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaac

SEQ ID NO: 52 - protein, 15 aa, synthetic construct
GLNDIFEAQKIEWHE

SEQ ID NO: 53 - protein, 26 aa, synthetic construct
KRRWKKNFIAVSAANRFKKISSSGAL

SEQ ID NO: 54 - protein, 6 aa, synthetic construct
EEEEEE

SEQ ID NO: 55 - protein, 13 aa, synthetic construct
GAPVPYPDPLEPR

SEQ ID NO: 56 - protein, 8 aa, synthetic construct
DYKDDDDK

SEQ ID NO: 57 - protein, 9 aa, synthetic construct
YPYDVPDYA

SEQ ID NO: 58 - protein, 6 aa, synthetic construct
HHHHHH

SEQ ID NO: 59 - protein, 10 aa, synthetic construct
EQKLISEEDL

SEQ ID NO: 60 - protein, 18 aa, synthetic construct
TKENPRSNQEESYDDNES

SEQ ID NO: 61 - protein, 15 aa, synthetic construct
KETAAAKFERQHMDS

SEQ ID NO: 62 - protein, 38 aa, synthetic construct
MDEKTTGWRGGHVVEGLAGELEQLRARLEHHPQGQREP

SEQ ID NO: 63 - protein, 13 aa, synthetic construct
SLAELLNAGLGGS

SEQ ID NO: 64 - protein, 8 aa, synthetic construct
TQDPSRVG

SEQ ID NO: 65 - protein, 8 aa, synthetic construct
WSHPQFEK

SEQ ID NO: 66 - protein, 6 aa, synthetic construct
CCPGCC

SEQ ID NO: 67 - protein, 14 aa, synthetic construct
GKPIPNPLLGLDST

SEQ ID NO: 68 - protein, 11 aa, synthetic construct
YTDIEMNRLGK

SEQ ID NO: 69 - protein, 8 aa, synthetic construct
DLYDDDDK

SEQ ID NO: 70 - protein, 16 aa, synthetic construct
TDKDMTITFTNKKDAE

SEQ ID NO: 71 - protein, 13 aa, synthetic construct
AHIVMVDAYKPTK

SEQ ID NO: 72 - protein, 12 aa, synthetic construct
KLGDIEFIKVNK

SEQ ID NO: 73 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL